(19) United States
(12) Patent Application Publication
(10) Pub. No.: US 2026/0226552 A1
(43) Pub. Date: Aug. 6, 2026

(54) MUC5AC IN PANCREATIC DUCTAL ADENOCARCINOMA

(71) Applicant: Ohio State Innovation Foundation, Columbus, OH (US)

(72) Inventor: Ashish MANNE, Columbus, OH (US)

(21) Appl. No.: 19/150,662

(22) PCT Filed: Jan. 24, 2024
(86) PCT No.: PCT/US24/12771

§ 371 Date: Jul. 24, 2025

Related U.S. Application Data
(60) Provisional application No. 63/481,349, filed on Jan. 24, 2023.

(51) Int. Cl. C12Q 1/6886; A61K 31/4745; A61K 31/513; A61K 31/519; A61K 31/555; A61K 31/7068; A61K 33/243; A61P 35/00; G01N 33/575; G01N 33/57525

(52) U.S. Cl. CPC C12Q 1/6886; A61K 31/4745; A61K 31/513; A61K 31/519; A61K 31/555; A61K 31/7068; A61K 33/243; A61P 35/00; G01N 33/57525; G01N 33/57585; C12Q 2600/106; C12Q 2600/156; G01N 2333/4725

(57) ABSTRACT

The present disclosure relates methods of using Mucin-5AC (MUC5AC) expression, location, and/or composition to treat, diagnose, predict, monitor, and/or track pancreatic ductal adenocarcinoma (PDA).

### CROSS-REFERENCE TO RELATED APPLICATION

This application claims priority to, and the benefit of, U.S. Provisional Patent Application No. 63/481,349, filed Jan. 24, 2023, entitled “MUC5AC in Pancreatic Ductal Adenocarcinoma,” which is incorporated by reference herein in its entirety.

### FIELD

The present disclosure provides methods of treating subjects with pancreatic ductal adenocarcinoma (PDA) by measuring MUC5AC dynamics. The present disclosure also provides methods of detecting, monitoring, predicting, and/or tracking PDA patient responses to cancer treatments using MUC5AC dynamics.

### BACKGROUND

Pancreatic ductal adenocarcinoma (PDA) is a lethal malignancy with an all-stage five-year survival rate of just 10%. Gemcitabine/nab-paclitaxel (NP) and FOLFIRINOX are two category 1 systemic therapy recommendations for PDA, and neither of them has proved to be superior in the perioperative or metastatic setting. In current clinical practice, age and performance status are key determinants for selecting a treatment regimen. Platinum-based therapy is preferred in patients with specific germline mutations (BRCA1/2, PALB2, ATM, and RAD50, and PALB2), but their prevalence is extremely low (<10% in sporadic cancers and up to 17% in familial cancer syndromes). Given the limitations of high mortality and limited treatment options for advanced PDA, there is a need to develop a method of predicting treatment response in PDA, and discovery of such a method will also open doors for targeted drug discovery.

The methods and treatments disclosed herein address these needs and more.

### SUMMARY

The present disclosure provides methods of treating subjects with a pancreatic lesion including, but not limited to benign pancreatic lesions, pre-cancerous pancreatic lesions, and malignant pancreatic lesions (such as, for example PDA), by detecting and/or scoring MUC5AC levels. The present disclosure also provides methods of monitoring and/or predicting treatment responses against a pancreatic lesion including, but not limited to benign pancreatic lesions, pre-cancerous pancreatic lesions, and malignant pancreatic lesions (such as, for example PDA), based on detecting and/or scoring MUC5AC levels alone or in combination with other PDA biomarkers including, but not limited to CA19-9.

In one aspect, disclosed herein is a method of treating a pancreatic lesion in a subject, the method comprising collecting a tissue sample from the subject, detecting Mucin-5AC (MUC5AC) levels in the tissue sample, classifying the subject as having a benign pancreatic lesion, a pre-cancerous pancreatic lesion, or a malignant pancreatic lesion, wherein i) the benign pancreatic lesion will not express MUC5AC, ii) the pre-cancerous pancreatic lesion comprises increased expression of the immature MUC5AC, the mature MUC5AC, or a combination thereof, relative to a healthy control or the benign pancreatic lesion, and iii) the malignant pancreatic lesion comprises increased expression of the immature MUC5AC, mature MUC5AC, or a combination thereof, relative to the benign pancreatic lesion or the pre-cancerous pancreatic lesion; and treating the subject classified as having the benign pancreatic lesion by monitoring said subject for progression or regression of the pancreatic lesion, treating the subject classified as having the pre-cancerous pancreatic lesion by monitoring said subject for progression or regression of the lesion, or surgical resection of the pancreatic lesion, and treating the subject classified as having the malignant pancreatic lesion with at least one anti-cancer therapy comprising a chemotherapeutic agent, an imaging modality, or surgical resection.

In one aspect, disclosed herein is a method of decreasing expression of Mucin-5AC (MUC5AC) in a malignant pancreatic cell in a subject diagnosed with pancreatic ductal adenocarcinoma (PDA), the method comprising collecting a tissue sample from the subject, detecting an expression level of MUC5AC in the sample relative to a control sample, and treating the subject with at least one anti-cancer therapy, wherein the anti-cancer therapy reduces the size of the PDA and the expression level of MUC5AC in the subject relative to the control.

In one aspect, disclosed herein is a method of preventing maturation of Mucin-5AC (MUC5AC) in a pancreatic cell in a subject diagnosed with a pancreatic lesion, the method comprising collecting a tissue sample from a subject, detecting an immature MUC5AC, a mature MUC5AC, or a combination thereof, in the tissue sample, and treating the subject with PDA with at least one anti-cancer therapy, wherein the anti-cancer therapy prevents the immature MUC5AC from converting into the mature MUC5AC or an immunogenic MUC5AC, or wherein the anti-cancer therapy prevents the mature MUC5AC from converting into the immunogenic MUC5AC.

In one aspect, disclosed herein is a method of monitoring a treatment response against a pancreatic ductal adenocarcinoma (PDA) in a subject, the method comprising collecting a first tissue sample from the subject, detecting and scoring MUC5AC in the first tissue sample, diagnosing the subject with a benign pancreatic lesion, a pre-cancerous pancreatic lesion, or a malignant pancreatic lesion, treating the subject with a first anti-cancer therapy when the subject is diagnosed with the pre-cancerous pancreatic lesion or the malignant pancreatic lesion, collecting a second tissue sample from the subject, detecting and scoring MUC5AC in the second tissue sample, and treating the subject with a second anti-cancer therapy when MUC5AC increases in the subject relative to a control, or continuing with first anti-cancer therapy MUC5AC decreases or remains unchanged in the subject relative to a control.

In one aspect, disclosed herein is a method of monitoring a treatment response against a pancreatic ductal adenocarcinoma (PDA) in a subject, the method comprising collecting a first tissue sample from the subject, detecting and scoring a MUC5AC and CA19-9 in the first tissue sample, diagnosing the subject with a benign pancreatic lesion, a pre-cancerous pancreatic lesion, or a malignant pancreatic lesion, treating the subject with a first anti-cancer therapy when the subject is diagnosed with the pre-cancerous pancreatic lesion or the malignant pancreatic lesion, collecting a second tissue sample from the subject, detecting and scoring MUC5AC and CA19-9 in the second tissue sample, and treating the subject with a second anti-cancer therapy when MUC5AC and CA19-9 increase in the subject relative to a control, or continuing with first anti-cancer therapy when MUC5AC remains unchanged or decreases and CA19-9 decreases in the subject relative to a control.

In some embodiments, the method of any preceding aspect comprises a benign pancreatic lesion comprising a low-risk for PDA. In some embodiments, the method of any preceding aspect comprises a pre-cancerous pancreatic lesion comprising a moderate-risk for PDA. In some embodiments, the method of any preceding aspect comprises a malignant pancreatic lesion comprising a high-risk for PDA. In some embodiments, the malignant pancreatic lesion comprises a PDA.

In some embodiments, the method of any preceding aspect comprises a scoring system of MUC5AC levels, wherein MUC5AC levels are scored from 0 to 300. In some embodiments, the benign pancreatic lesion comprises a MUC5AC score of 50 or less. In some embodiments, the pre-cancerous pancreatic lesion comprises a MUC5AC score of 100 or less. In some embodiments, the malignant pancreatic lesion comprises a MUC5AC score greater than 100. In some embodiments, the healthy control comprises a MUC5AC score less than 10.

In some embodiments, the tissue sample (including, but not limited to a first, second, or third tissue sample) comprises a blood sample, a serum sample, a plasma sample, or a pancreatic tissue biopsy. In some embodiments, a first, second, third, or more tissue samples are collected at least 24 hours apart.

In some embodiments, the method of any preceding aspect detects MUC5AC by an antibody, or a fragment thereof, an enzyme linked immunosorbent assay (ELISA), a western blot assay, mass spectrometry, RNA sequencing, real-time polymerase chain reaction (RT-PCR), or a derivative thereof. In some embodiments, the antibody, or a fragment thereof, comprises a monoclonal antibody including, but not limited to CLH2, 45M1, I-131, 2-11M1, Nd2, or a fragment thereof.

In some embodiments, the method of any preceding aspect further detects carbohydrate antigen 19-9 (CA19-9), a methylation biomarker, a blood biomarker, or a combination thereof. In some embodiments, the methylation marker includes, but is not limited to ADAMTS1, ADAMTS22, ALX4, APC, BMP3, BNC1, BRCA1, CCND2, CDKN1C, CUX2, DAPKI, DCC, EPB41L3, ESR1, FAM150A, FSD1, GPC3, HIC1, HIST1H4E, HOXA1, MAPT, LOC100128977, LOC100130148, MESTv2, MIR663, MUC2, MYF3, MYOD1, NPTX2, p14, p16, PCDH10, PENK, PGKI, PGR-dist, PGR-prox, PLAU, ppENK, RARB, RASSFlA, REGlA, RUNX3, SARP2, SEMA5A, SEPT9v2, SFRP1, SFRP2, SIX3, SOCS1, SPARC, SPSB4, SRBC, SST, SYK, TACl, TBX3, TFPI2, THBS1, TMS, TRIM73, TSPAN2, UCLH1, VHL, WNT5A, ZNF154, ZNF695, hMLH1, CDKN2B, RB1, or a combination thereof. In some embodiments, the blood biomarker comprises a mutation to one or more genes selected from KRAS, p53, CDK2NA, ATM, and PIK3CA.

In some embodiments, the anti-cancer therapy (including, but not limited to a first, second, or third anti-cancer therapy) comprises a chemotherapeutic agent, an imaging modality, surgical resection, or a combination thereof. In some embodiments, the chemotherapeutic agent includes, but is not limited to fluorouracil (5FU), cisplatin, irinotecan, oxaliplatin, gemcitabine (Gem), nab-paclitaxel, leucovorin, or a combination thereof. In some embodiments, the imaging modality includes, but is not limited to magnetic resonance imaging (MRI), computed tomography (CT), positron emission tomography (PET), endoscopic ultrasound (EUS), abdominal ultrasound, or a combination thereof.

In some embodiments, the method of any preceding aspect inhibits secretion of MUC5AC from a malignant pancreatic cell.

### BRIEF DESCRIPTION OF FIGURES

The accompanying figures, which are incorporated in and constitute a part of this specification, illustrate several aspects described below.

FIG. 1 shows MUC5AC expression in pancreatic cancer progression. In normal pancreatic cell, MUC5AC production is repressed and is initiated when there is an appropriate insult. Depending on the nature of insult, the composition and therefore the localization, and the level of MUC5AC produced may differ. If the insult has malignant potential, higher levels of mature MUC5AC with abnormal sorting ensues. Aggressive tumors with poor treatment response and worse outcome tend to have more apical and inter-cellular mature MUC5AC compared to the favorable tumors. If the insults lead to pre-cancerous or benign conditions, more immature MUC5AC with controlled sorting occurs, and during malignant transformation MUC5AC composition and localization changes.

FIG. 2 shows the characteristics and features of the MUC5AC signature.

FIG. 3 shows diagnosis of pancreatic ductal adenocarcinoma. 1—refers to the methylation markers in Table 1. 2—refers to the combination of mature MUC5AC including NPC-1C and PAM4 reactive MUC5AC and immature MUC5AC by enzyme-linked immunoassay (ELISA). 3—refers to along with high intensity of MUC5AC in the apical or extracellular region compared to peri-nuclear region. 4—refers to along with higher intensity on the peri-nuclear region compared to apical or extracellular region. WGA refers to wheat germ agglutinin assay. IHC refers to immunohistochemistry.

FIG. 4 shows the screening for PDA. 1—refers to the methylation markers in Table 1. 2—refers to the combination of mature MUC5AC including NPC-1C and PAM4 reactive MUC5AC and immature MUC5AC by enzyme-linked immunoassay (ELISA). 3—refers to along with high intensity of MUC5AC in the apical or extracellular region compared to peri-nuclear region. 4—refers to along with higher intensity on the peri-nuclear region compared to apical or extracellular region. WGA refers to wheat germ agglutinin assay. IHC refers to immunohistochemistry.

FIG. 5 shows the use of MUC5AC signature alone in diagnosing PDA.

FIG. 6 shows the relationship between MUC5AC and CA19-9 with tumor burden and treatment response.

FIG. 7 shows the serum MUC5AC and CA 19-9 levels in patients responding to therapy.

FIG. 8 shows the serum MUC5AC and CA 19-9 levels in patients not responding to therapy.

FIG. 9 shows the correlation between MUC5AC and CA19-9 levels.

FIG. 10 shows the MUC5AC distribution in the resected pancreatic tumors. Blue —neoadjuvant therapy group, Red—upfront surgery group.

FIG. 11 shows the treatment response and MUC5AC expression in all patients (N=100). *Objective response (OR) vs. Upfront surgery (UpS)—p=0.04; ^{#}OR vs. UpS-p=0.01; \hat{ }OR vs. Ups-p=0.01.

FIG. 12 shows a summary of samples used herein.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “screening” refers to a method especially used in drug discovery in which data processing/control software, liquid handling devices, and sensitive detectors can allow for quick conductions of chemical, genetic, or pharmacological tests. This process allows one to quickly recognize active compounds, antibodies, or genes that modulate a particular biomolecular pathway. The results of these processes provide starting points for drug design.

As used herein, “monitoring” refers to the actions of observing and checking the progress or quality of a treatment or procedure over a period of time. “Monitoring” also refers to observing the course of a disease or condition, such as a cancer, over a period of time.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “control” is an alternative subject or sample used in an experiment for comparison purposes. A control can be “positive” or “negative.”

As used herein, “normal” refers to a standard of health where no disease, disorder, abnormal condition, mutation, or dysfunction exists within a particular subject or patient. “Normal” can also refer to an average or typical state or condition.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

“Expression” as used herein refers to the process by which information from a gene is used in the synthesis of a functional gene product that enables it to produce a peptide/protein end product, and ultimately affect a phenotype, as the final effect.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely a adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

The terms “immunotherapy” and “immunotherapeutic” refers to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

The terms “anticancer” and “anticarcinogen” refers to a substance, composition, or formula that counteracts the effects or inhibits the development of a cancerous cells and tissues.

The term “cancer” is used to address any neoplastic disease, and is not limited to epithelial neoplasms (surface and glandular cancers; such a squamous cancers or adenomas). It is used here to describe both solid tumors and hematologic malignancies, including epithelial (surface and glandular) cancers, soft tissue and bone sarcomas, angiomas, mesothelioma, melanoma, lymphomas, leukemias and myeloma.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of cancer), during early onset (e.g., upon initial signs and symptoms of cancer), or after an established development of cancer.

As used herein, a “therapeutic regimen” refers to a structured treatment plan or strategy designed to improve and maintain health. Generally, a therapeutic regimen will be designed, prescribed, and/or administered by a licensed medical practitioner. The therapeutic regimen generally specifies the treatment dosage, the treatment scheduling, and the duration of the treatment. In some embodiments, the therapeutic regimen comprises one or more therapeutic compositions. In some embodiments, the therapeutic regimen comprises one or more therapeutic agents. In some embodiments, the therapeutic regimen comprises any combination of therapeutic compositions and therapeutic agents, such as for example the combination of an inhibitor and an antibody. In some embodiments, a therapeutic regimen comprises modifying, continuing, and/or initiating at least one therapeutic agent and/or therapeutic composition. In some embodiments, a therapeutic regimen comprises treating and/or preventing a disease, disorder, and/or condition.

Methods of Predicting and/or Treating Pancreatic Ductal Adenocarcinomas (PDAs)

PDAs represent a cancer entity with extraordinarily high malignancy, poor prognosis, and constantly increasing patient numbers. Its aggressive nature and the fact that majority patients only present symptoms once in advanced stages of PDA render the development of PDA therapies extremely challenging. In addition, there are also limitations to the early diagnosis of PDA, where the prevalence of biomarkers, including, but not limited to serum biomarkers, methylation markers, and DNA mutation markers, are too low to reliably predict PDA development and progression. Thus, there remains a need to identify and implement a novel PDA biomarker that can reliably predict PDA development and progression. Further, there is a need for a novel PDA biomarker that can be combined with current diagnostic techniques, such as biomarkers, tumor imaging, and tissue biopsies, to optimally predict PDA development and progression.

Initial methods of treating PDA involved single agent therapies that lagged behind the expectation of PDA regression. Later advancements in pancreatic cancer management identified combination therapies, such as for example tumor resection and chemotherapy, as the optimal curative treatment option. However, the majority of PDA patients still develop disease recurrence within 2 years of receiving initial treatment. Thus, indicating that predicting patient responses to treatments is lacking. Many factors associated with PDA recurrence have been identified, such as age, preoperative serum CA19-9 levels, vascular resection, perivascular invasion, lymph node status and ratio, and tumor size/stage, however these prognostic factors are not specific to pancreatic tumors nor are they reliable for long term (i.e. greater than 2 years) prognosis following therapy. Thus, there is a need to identify PDA-specific prognostic marker that can be predictive of patient treatment responses and long term PDA prognosis.

Mucin-5AC (MUC5AC) is a large gel-forming glycoprotein encoded by the MUC5AC gene found in the respiratory airway secretions pooled from healthy humans. MUC5AC exists as 2 isoforms: immature MUC5AC and mature MUC5AC, wherein the immature MUC5AC is an unglycosylated monomer, and the mature MUC5AC is heavily glycosylated oligomer. Immunogenic MUC5AC also exists as a subtype of mature MUC5AC that is heavily glycosylated, and comprises an epitope capable of eliciting an immune response. When any isoform or variant of MUC5AC is expressed in pancreas, it is often associated with pancreatic pathology, including PDA. In benign pancreatic lesions and/or early stages of PDA, MUC5AC is present as the immature monomer, remaining localized within the pancreatic cells. In advanced PDA, MUC5AC is a highly glycosylated, oligomerized protein that is secreted from the pancreas into extracellular spaces. Thus, presenting a novel biomarker for diagnosis, prognosis, and treatment of PDA. Therefore, the present disclosure demonstrates that MUC5AC dynamics in the pancreas correlates with the development, progression, and/or metastases of PDA.

The present disclosure provides methods of treating subjects with a pancreatic lesion including, but not limited to benign pancreatic lesions, pre-cancerous pancreatic lesions, and malignant pancreatic lesions (such as, for example PDA), by detecting and/or scoring MUC5AC levels. The present disclosure also provides methods of monitoring and/or predicting treatment responses against a pancreatic lesion including, but not limited to benign pancreatic lesions, pre-cancerous pancreatic lesions, and malignant pancreatic lesions (such as, for example PDA), based on detecting and/or scoring MUC5AC levels alone or in combination with other PDA biomarkers including, but not limited to CA19-9.

In one aspect, disclosed herein is a method of treating a pancreatic lesion in a subject, the method comprising collecting a tissue sample from the subject, detecting Mucin-5AC (MUC5AC) levels in the tissue sample, classifying the subject as having a benign pancreatic lesion, a pre-cancerous pancreatic lesion, or a malignant pancreatic lesion, wherein i) the benign pancreatic lesion comprises expression of an immature MUC5AC, a mature MUC5AC, or a combination thereof, relative to a healthy control, ii) the pre-cancerous pancreatic lesion comprises increased expression of the immature MUC5AC, the mature MUC5AC, or a combination thereof, relative to a healthy control or the benign pancreatic lesion, and iii) the malignant pancreatic lesion comprises increased expression of the immature MUC5AC, mature MUC5AC, or a combination thereof, relative to the benign pancreatic lesion or the pre-cancerous pancreatic lesion; and treating the subject classified as having the benign pancreatic lesion by monitoring said subject for progression or regression of the pancreatic lesion, treating the subject classified as having the pre-cancerous pancreatic lesion by monitoring said subject for progression or regression of the lesion, or surgical resection of the pancreatic lesion, and treating the subject classified as having the malignant pancreatic lesion with at least one anti-cancer therapy comprising a chemotherapeutic agent, an imaging modality, or surgical resection.

In one aspect, disclosed herein is a method of decreasing expression of Mucin-5AC (MUC5AC) in a malignant pancreatic cell in a subject diagnosed with pancreatic ductal adenocarcinoma (PDA), the method comprising collecting a tissue sample from the subject, detecting an expression level of MUC5AC in the sample relative to a control sample, and treating the subject with at least one anti-cancer therapy, wherein the anti-cancer therapy reduces the PDA and the expression level of MUC5AC in the subject relative to the control.

In some embodiments, the method of any preceding aspect decreases MUC5AC expression by at least 10%. In some embodiments, the method of any preceding aspect decreases MUC5AC expression by 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, less than, greater than, or in between the given percentages relative to an untreated control. It should be noted that the method of any preceding aspect can decrease MUC5AC RNA and/or MUC5AC protein.

In one aspect, disclosed herein is a method of preventing maturation of Mucin-5AC (MUC5AC) in a pancreatic cell in a subject diagnosed with a pancreatic lesion, the method comprising collecting a tissue sample from a subject, detecting an immature MUC5AC, a mature MUC5AC, or a combination thereof, in the tissue sample, and treating the subject with PDA with at least one anti-cancer therapy, wherein the anti-cancer therapy prevents the immature MUC5AC from converting into the mature MUC5AC or an immunogenic MUC5AC, or wherein the anti-cancer therapy prevents the mature MUC5AC from converting into the immunogenic MUC5AC.

In one aspect, disclosed herein is a method of monitoring a treatment response against a pancreatic ductal adenocarcinoma (PDA) in a subject, the method comprising collecting a first tissue sample from the subject, detecting and scoring MUC5AC in the first tissue sample, diagnosing the subject with a benign pancreatic lesion, a pre-cancerous pancreatic lesion, or a malignant pancreatic lesion, treating the subject with a first anti-cancer therapy when the subject is diagnosed with the pre-cancerous pancreatic lesion or the malignant pancreatic lesion, collecting a second tissue sample from the subject, detecting and scoring MUC5AC in the second tissue sample, and treating the subject with a second anti-cancer therapy when MUC5AC increases in the subject relative to a control, or continuing with first anti-cancer therapy MUC5AC decreases or remains unchanged in the subject relative to a control.

In one aspect, disclosed herein is a method of monitoring a treatment response against a pancreatic ductal adenocarcinoma (PDA) in a subject, the method comprising collecting a first tissue sample from the subject, detecting and scoring a MUC5AC and CA19-9 in the first tissue sample, diagnosing the subject with a benign pancreatic lesion, a pre-cancerous pancreatic lesion, or a malignant pancreatic lesion, treating the subject with a first anti-cancer therapy when the subject is diagnosed with the pre-cancerous pancreatic lesion or the malignant pancreatic lesion, collecting a second tissue sample from the subject, detecting and scoring MUC5AC and CA19-9 in the second tissue sample, and treating the subject with a second anti-cancer therapy when MUC5AC and CA19-9 increase in the subject relative to a control, or continuing with first anti-cancer therapy when MUC5AC remains unchanged or decreases and CA19-9 decreases in the subject relative to a control.

As used herein, “benign pancreatic lesions” refers to nonreactive, precursor lesions commonly confined to the pancreas. Patients with benign lesions are commonly asymptomatic. Other diseases, including, but not limited to chronic pancreatitis (CP), autoimmune pancreatitis (AIP), and paraduodenal pancreatitis (PDP), are often mislabeled as, but often present similarly to benign pancreatic lesions. It should be understood that benign pancreatic lesions encompasses other pancreatic pathologies including, but not limited to CP, AIP, and PDP. Benign pancreatic lesions are commonly associated with low-risk of PDA.

As used herein, “pre-cancerous pancreatic lesions” refers to reactive pancreatic lesions that include pancreatic intraepithelial lesions, mucinous cystic neoplasms, and intraductal papillary neoplasms. Patients with pre-cancerous pancreatic lesions tend to begin showing mild to chronic symptoms, including, but not limited to painless jaundice, anorexia, weight loss, abdominal pain, and/or pancreatic endocrine & exocrine insufficiency. Pre-cancerous pancreatic lesions are commonly associated with moderate-risk of PDA.

As used herein, “malignant pancreatic lesions” refers to invasive, reactive pancreatic lesions that develop increasing genetic variability and proliferative characteristics that allow for eventual invasion and metastasis into other tissues. Malignant pancreatic lesions often causes structural deformity to the pancreas including abrupt obstruction of pancreatic ducts, obstruction of the common bile duct and/or the pancreatic duct, and tissue atrophy proximal (near) to the lesion(s). Patients with malignant pancreatic lesions often present with chronic fatigue, anorexia, weight loss, abdominal pain, jaundice, nausea, back pain, diarrhea, and/or vomiting. Malignant pancreatic lesions are commonly associated with high-risk PDA. In some embodiments, malignant pancreatic lesions comprise PDA.

In some embodiments, the method of any preceding aspect comprises a benign pancreatic lesion comprising a low-risk for PDA. In some embodiments, the method of any preceding aspect comprises a pre-cancerous pancreatic lesion comprising a moderate-risk for PDA. In some embodiments, the method of any preceding aspect comprises a malignant pancreatic lesion comprising a high-risk for PDA. In some embodiments, the malignant pancreatic lesion comprises a PDA.

In some aspects, the present disclosure provides methods of combining MUC5AC and at least one additional biomarker to detect, inhibit, regress, decrease, or prevent a pancreatic lesion in a subject. In some aspects, the present disclosure also provides methods of combining MUC5AC and at least one additional biomarker to predict, monitor, track, and/or observe anti-cancer treatment responses in a subject with a pancreatic lesion.

In some embodiments, the method of any preceding aspect further comprises detecting carbohydrate antigen 19-9 (CA19-9), a methylation biomarker, a blood biomarker, or a combination thereof. In some embodiments, the method of any preceding aspect comprises detecting and scoring MUC5AC and CA19-9 levels in a liquid tissue sample from the subject, wherein the method diagnoses, monitors, and/or treats a pancreatic lesion in the subject.

In some embodiments, the method of any preceding aspect comprises a scoring system (such as, for example an H scoring system) of MUC5AC levels. In some embodiments, the expression level of MUC5AC (including MUC5AC RNA and/or MUC5AC protein) of any preceding aspect is converted into a scoring system, wherein healthy controls score are at the lower end, malignant PDA scores are at the upper end, and benign PDA and pre-cancerous PDA comprise intermediate scores, and wherein pre-cancerous PDA scores are closer to the upper end relative to benign PDA. In some embodiments, the lower end is zero or one. In some embodiments, the upper end is 300, 500, 1000, or more. A non-limiting examples of the scoring system is where MUC5AC levels are scored from 0 to 300, 0 to 400, 0 to 500, 0 to 600, 0 to 700, 0 to 800, 0 to 900, or 0 to 1000.

In some embodiments, when using the 0 to 300 scoring system, the benign pancreatic lesion comprises a MUC5AC score of 50 or less. In some embodiments, when using the 0 to 300 scoring system, the benign pancreatic lesion comprises a MUC5AC score from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. In some embodiments, when using the 0 to 300 scoring system, the pre-cancerous pancreatic lesion comprises a MUC5AC score of 100 or less. In some embodiments, when using the 0 to 300 scoring system, the pre-cancerous pancreatic lesion comprises a MUC5AC score from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100. In some embodiments, when using the 0 to 300 scoring system, the malignant pancreatic lesion comprises a MUC5AC score greater than 100. In some embodiments, when using the 0 to 300 scoring system, the malignant pancreatic lesion comprises a MUC5AC score from 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, and 300. In some embodiments, when using the 0 to 300 scoring system, the healthy control comprises a MUC5AC score less than 10. In some embodiments, when using the 0 to 300 scoring system. The healthy control comprises a MUC5AC score from 0, 1, 2, 3, 4, 5, 6, 7, 8, 9, and 10.

In some embodiments, the tissue sample (including, but not limited to a first, second, or third tissue sample) include, but is not limited to a blood sample, a serum sample, a plasma sample, or a pancreatic tissue biopsy. In some embodiments, a first, second, third, or more tissue samples are collected at least 24 hours apart. In some embodiments, the first, second, third, or more tissue samples are collected 24, 36, 48, 60, 72, 84, 96, 120, 150, 180, 200, 240, 250, 270, 300, 350, 400, 450, 500 hours apart, or any number of hours more, less, or any number in between the given range. In some embodiments, the first, second, third, or more tissue samples are collected 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365 days or more apart.

In some embodiments, the method of any preceding aspect detects MUC5AC by an antibody, or a fragment thereof, an enzyme linked immunosorbent assay (ELISA), a western blot assay, mass spectrometry, RNA sequencing, real-time polymerase chain reaction (RT-PCR), or a derivative thereof. In some embodiments, the antibody, or a fragment thereof, comprises a monoclonal antibody including, but not limited to CLH2, 45M1, 2-11M1, Nd2, or a fragment thereof.

In some embodiments, the method of any preceding aspect further detects carbohydrate antigen 19-9 (CA19-9), a methylation biomarker, a blood biomarker, or a combination thereof. In some embodiments, the methylation marker includes, but is not limited to ADAMTS1, ADAMTS22, ALX4, APC, BMP3, BNC1, BRCA1, CCND2, CDKN1C, CUX2, DAPKI, DCC, EPB41L3, ESR1, FAM150A, FSD1, GPC3, HIC1, HIST1H4E, HOXA1, MAPT, LOC100128977, LOC100130148, MESTv2, MIR663, MUC2, MYF3, MYOD1, NPTX2, p14, p16, PCDH10, PENK, PGKI, PGR-dist, PGR-prox, PLAU, ppENK, RARB, RASSFlA, REGlA, RUNX3, SARP2, SEMA5A, SEPT9v2, SFRP1, SFRP2, SIX3, SOCS1, SPARC, SPSB4, SRBC, SST, SYK, TAC1, TBX3, TFPI2, THBS1, TMS, TRIM73, TSPAN2, UCLH1, VHL, WNT5A, ZNF154, ZNF695, hMLH1, CDKN2B, RB1, or a combination thereof. In some embodiments, the blood biomarker comprises a mutation to one or more genes selected from KRAS, p53, CDK2NA, ATM, and PIK3CA. In some embodiments, CA19-9, the methylation biomarker, the blood biomarker are detected in a tissue sample including, but not limited to a blood sample, a serum sample, a plasma sample, or a pancreatic tissue biopsy.

In some embodiments, the anti-cancer therapy (including, but not limited to a first, second, or third anti-cancer therapy) comprises a chemotherapeutic agent, an imaging modality, surgical resection, or a combination thereof. In some embodiments, the chemotherapeutic agent includes, but is not limited to fluorouracil (5FU), cisplatin, irinotecan, oxaliplatin, gemcitabine (Gem), nab-paclitaxel (NP), leucovorin, or a combination thereof. In some embodiments, the imaging modality includes, but is not limited to magnetic resonance imaging (MRI), computed tomography (CT), positron emission tomography (PET), endoscopic ultrasound (EUS), abdominal ultrasound, or a combination thereof.

In some embodiments, the method of any preceding aspect inhibits secretion of MUC5AC from a malignant pancreatic cell.

In some embodiments, the PDA of any preceding aspect or the pancreatic lesion of any preceding aspect comprises greater than 0% increase in MUC5AC expression relative to a healthy control. In some embodiments, the PDA of any preceding aspect or the pancreatic lesion of any preceding aspect increases MUC5AC expression by 0.25%, 0.50%, 0.75%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, less than, greater than, or in between the given percentages relative to a healthy control.

In some embodiments, the method of any preceding aspect prevents glycosylation of MUC5AC in normal pancreatic tissues benign pancreatic lesion, and pre-cancerous pancreatic lesion. In some embodiments, the method of any preceding aspect, reverses the glycosylation of MUC5AC, wherein any number of MUC5AC proteins comprise reduced glycosyl moieties. In some embodiments, the method of any preceding aspect, prevents or reverse oligomerization of immature MUC5AC. As used herein, “oligomerization” refers to a biochemical process that converts monomers into macromolecular complexes, also known as oligomers, through a finite degree of polymerization, or chemically binding small molecules to create larger molecules.

In one aspect, disclosed herein is a method of predicting an anti-cancer therapy response in a subject with a pancreatic lesion, the method comprising collecting a sample from the subject, detecting a MUC5AC in the sample, diagnosing the subject with a benign pancreatic lesion, a pre-cancerous pancreatic lesion, or a malignant pancreatic lesion, and assessing which treatment will reduce, decrease, and regress the pancreatic lesion without recurrence, wherein the treatment comprises fluorouracil (5FU), Cisplatin, Irinotecan, Oxaliplatin, Gemcitabine (Gem), Nab-paclitaxel (NP), Leucovorin, FOLFIRINOX, an imaging modality, tumor resection, or a combination thereof.

In one aspect, disclosed herein is a method of tracking or recording an anti-cancer therapy response in a subject with a pancreatic lesion, the method comprising collecting a first sample from the subject, detecting a MUC5AC in the first sample, diagnosing the subject with a benign pancreatic lesion, a pre-cancerous pancreatic lesion, or a malignant pancreatic lesion, treating the subject with a first anti-cancer therapy, collecting at least one additional sample from the subject, and detecting a MUC5AC in the at least one additional sample, wherein when MUC5AC levels in the at least one additional sample are decreasing relative MUC5AC levels in the first sample the first anti-cancer therapy is successful, or wherein MUC5AC levels in the at least one additional sample are increasing relative MUC5AC levels in the first sample a second anti-cancer therapy is administered.

As used herein, “progression” or “cancer progression” refers to the process by which the state of the PDA or any other cancer worsens over time. The progression is characterized by enlargement of the original tumor, new metastasis to other tissues including lymph nodes, or disease recurrence.

As used herein, “regression” or “cancer regression” refers to the partial or complete disappearance of a tumor (benign or malignant) in the absence or presence of treatment. It should be noted that regression and remission can be used interchangeably.

As used herein, “treatment response” refers to the extent or degree to which a patient improves irrespective of the presence or absence of symptoms. In optimal situations, the treatment response of a patient can improve by 5%, 10%, 15%, 20%, 25%, 30%, 35%, 40%, 45%, 50%, 55%, 60%, 65%, 70%, 75%, 80%, 85%, 90%, 95%, 99%, or 100%. In suboptimal situations, the treatment response of a patient can worsen by 5%, 10%, 15%, 20%, 25%, 30%, 35%, 40%, 45%, 50%, 55%, 60%, 65%, 70%, 75%, 80%, 85%, 90%, 95%, 99%, or 100%. In some situations, the treatment response may not improve or worsen.

In some embodiments, the method of any preceding aspect further comprises administering to the subject an additional therapeutic agent to achieve the desired result of pancreatic lesion regression or remission, and alleviating other symptoms experienced by the subject. In some embodiments, the additional therapeutic agent comprises an antibiotic including, but not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine); an anti-inflammatory compound including, but is not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, mycophenolate; an anesthetic including, but is not limited to chloroprocaine, procaine, tetracaine, lidocaine, bupivacaine, ropivacaine, mepivacaine, levobupivacaine; a sedative including, barbiturates, benzodiazepines, nonbenzodiazepines hypnotics, antihistamines, muscle relaxants, opioids, methaqualone, or derivatives thereof. In some embodiments, the additional therapeutic agent of any preceding aspect is administered alone or in combination.

The anti-cancer therapy or therapeutic agent of any preceding aspect may be administered in such amounts, time, and route deemed necessary in order to achieve the desired result. The exact amount of the anti-cancer therapy or therapeutic agent of any preceding aspect will vary from subject to subject, depending on the species, age, and general condition of the subject, the severity of the PDA, the particular anti-cancer therapy or therapeutic agent of any preceding aspect, its mode of administration, its mode of activity, and the like. The anti-cancer therapy or therapeutic agent of any preceding aspect is preferably formulated in dosage unit form for ease of administration and uniformity of dosage. It will be understood, however, that the total daily usage of the anti-cancer therapy or therapeutic agent of any preceding aspect will be decided by the attending physician within the scope of sound medical judgment. The specific therapeutically effective dose level for any particular subject will depend upon a variety of factors including the PDA being treated and the severity of the PDA; the activity of the anti-cancer therapy or therapeutic agent of any preceding aspect employed; the specific anti-cancer therapy or therapeutic agent of any preceding aspect employed; the age, body weight, general health, sex and diet of the patient; the time of administration, route of administration, and rate of excretion of the specific anti-cancer therapy or therapeutic agent of any preceding aspect employed; the duration of the treatment; drugs used in combination or coincidental with the specific anti-cancer therapy or therapeutic agent of any preceding aspect employed; and like factors well known in the medical arts.

The anti-cancer therapy or therapeutic agent of any preceding aspect may be administered by any route. In some embodiments, the anti-cancer therapy or therapeutic agent of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the anti-cancer therapy or therapeutic agent of any preceding aspect (e.g., its stability in the environment of the gastrointestinal tract), the condition of the subject (e.g., whether the subject is able to tolerate administration), etc.

The exact amount of anti-cancer therapy or therapeutic agent of any preceding aspect required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the method of any preceding aspect further comprises visualizing a pancreatic tissue using an imaging modality. In some embodiments, the imaging modality comprises magnetic resonance imaging (MRI), computed tomography (CT), positron emission tomography (PET), endoscopic ultrasound (EUS), abdominal ultrasound, or a combination thereof.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: MUC5AC in Pancreatic Ductal Adenocarcinoma

The existing biomarker landscape for PDA is limited, and there are no reliable diagnostic, prognostic and predictive biomarkers. Patients often present with painless jaundice or abdominal/back pain or incidentally on imaging. There are no robust guidelines for screening PDA, but in patients with a strong family history of germline cancers such as Lynch, Magnetic resonance imaging (MRI) and Endoscopic ultrasound (EUS) is advised. Predicting the malignancy of intraductal papillary mucinous neoplasm of the pancreas (IPMN) or other cystic lesions in pancreatic tissue is challenging. Current standard is to proceed with sampling of the lesion with invasive procedures such as EUS in lesions with perceived high-risk based on the size, imaging features and location of the lesions. Carbohydrate antigen 19-9 (CA 19-9) in blood is not a reliable diagnostic or screening marker as it can vary with other disease conditions.

In diagnosed patients on therapy, periodic imaging and serum CA19-9 are used to monitor response to treatments. Detection of microsatellite instability (for immunotherapy) and BRCA 1/2 (PARP inhibitor) and other DNA damage repair gene mutations (platinum chemotherapy) help in choosing therapy, but the prevalence of such markers is very low. In the experimental setting, some predictive markers in tumor tissue such as human equilibrate nucleoside transporter 1 (hENTI), secreted protein acidic and rich in cysteine (SPARC), epigenetic markers (GSTM1, ONECUT2, TET1, and MGMT), and circulating tumor DNA detection have been studied, but none of these are routinely used in a clinical practice.

Mucin-5AC (MUC5AC) is a gel-forming, glycosylated, high-molecular-weight protein expressed in abnormal pancreatic tissues, including PDA. Its detection in surface mucosal cells in stomach and bronchial tract is normal, but not in pancreatic tissues. There are MUC5AC isoforms and there is evidence on the prognostic value of mature MUC5AC (detected by the CLH2 monoclonal antibody [mab]), which are inconclusive. Sequential post-transcriptional changes occur as MUC5AC translocates from the perinuclear region and moves to the apical cell surface, including dimerization of unglycosylated MUC5AC monomer, the addition of N-acetyl galactosamine residues (maturation by glycosylation), multimerization, and finally, secretion of mature MUC5AC into the duct or inter-cellular regions. In pancreatic cells, MUC5AC modifications, specifically N-glycosylation, have been shown to promote carcinogenesis via multiple pathways (TGF-β, TNF, NF-kappa-B) and TFEB-related lysosomal changes.

### Glycosylation, Localization, and Immunoreactivity of MUC5AC

MUC5AC isoforms can be broadly divided into immature and mature MUC5AC isoforms. Immature MUC5AC is the initial unglycosylated (or minimally glycosylated) isoform in the perinuclear region. It can be detected by CLH2 mab. Mature MUC5AC is a heavily glycosylated MUC5AC variant detected by mabs 45M1 or 2-11M1 or Nd2, and are localized primarily in apical, extracellular (secreted or inter-cellular). When subjected to growth factors, pancreatic cell lines (PCLs) produced more mature than immature MUC5AC isoforms, showing the difference in their functionality and malignancy. Immunogenic MUC5AC refers to a sub-type of mature MUC5AC variant with an epitope capable of eliciting immune reaction detected by NPC-1C and PAM4 mabs. These two variants are expressed only in malignant pancreatic (and colon cancer), but not in normal pancreatic tissues. The prevalence of NPC-1C and PAM4 in PDA ranges from 48-79% and 85% respectively.

Control over MUC5AC ‘sorting’ (distribution to apical vs. intercellular vs. perinuclear) is lost in lung cancers, cholangiocarcinoma, and PDA, where MUC5AC is misexpressed. Building upon the aberrant sorting concept, it is contemplated that a sequence of events that lead to the malignant transformation of pancreatic cells, the acceleration of malignant disease, and the reflection of this process is a measurable MUC5AC isoform signature.

### The MUC5AC Isoform Signature.

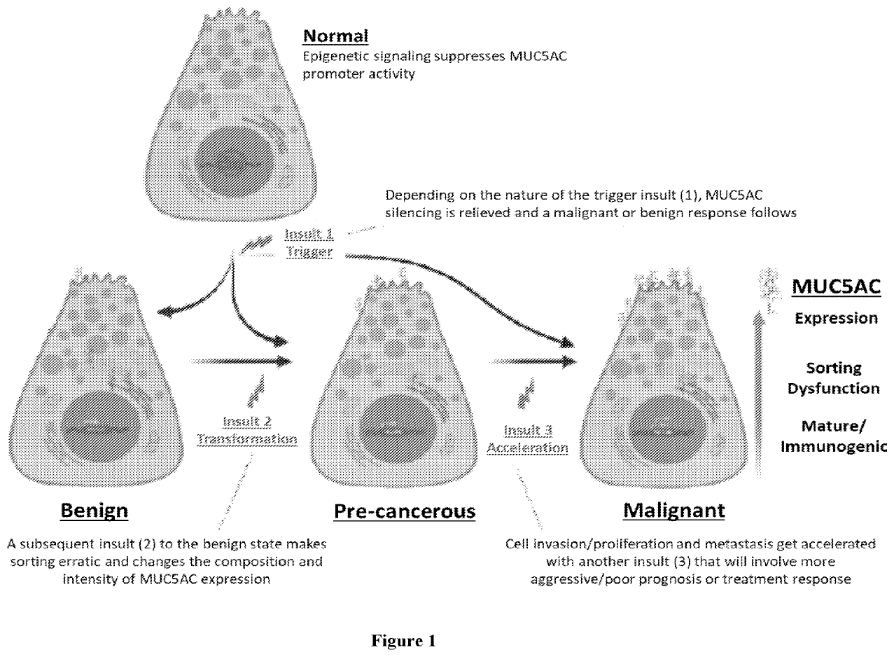

The series of events leading to pancreatic cell transformation are broadly divided into three inter-linked stages: 1) trigger response, 2) malignant transformation, and 3) malignant acceleration. These stages are, in turn, each defined by three components: a) MUC5AC sorting, b) MUC5AC isoform composition, and c) MUC5AC expression level. There is no evidence indicating that MUC5AC initiates the malignant process, but instead, it promotes malignant transformation and acceleration of metastasis (FIG. 1).

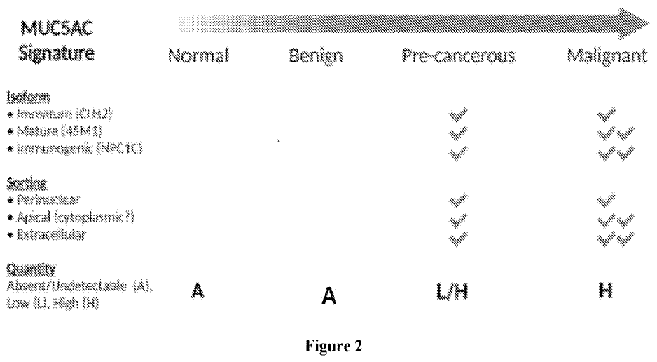

The MUC5AC signature model can account for the detection of MUC5AC in benign, pre-cancerous, and cancerous lesions and the differential pattern of expression and isoforms of MUC5AC in cancerous vs. pre-cancerous cells, and this disclosure provides the foundation to this concept with a focus on PDA biomarker development (FIG. 2).

In previous studies, outcomes were not correlated with MUC5AC isoform composition, localization, and degree of expression, which can explain the wide range of expression-outcome relationships. None of them could report isoform composition. Sorting could not be studied as most of them reported cytoplasmic staining, not apical/extracellular staining. As all these studies uses CLH2 mab that reacts to mature isoform, detection of mature isoforms (45M1 and NPC-1C) could give us more reliable information. The expression thresholds to classify the PDAs were different in these studies, making cross-study comparisons difficult to interpret. When the threshold was low (5 or 10%) for defining positive/negative, outcomes were better with MUC5AC detection in PDA. However, when the threshold was high (25% or high-H score), the outcomes were poor with its detection. Together, these limited and partially overlapping data underscore the importance of all three components in the MUC5AC signature.

The MUC5AC signature is a good candidate surrogate marker to diagnose PDA and predict treatment response, thereby helping physician treatment decision-making.

### MUC5AC Signature in Diagnosing PDA

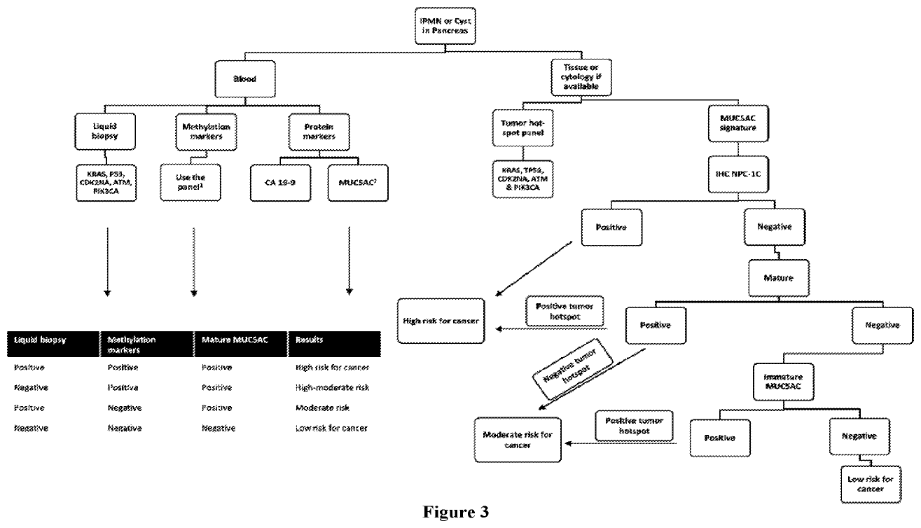

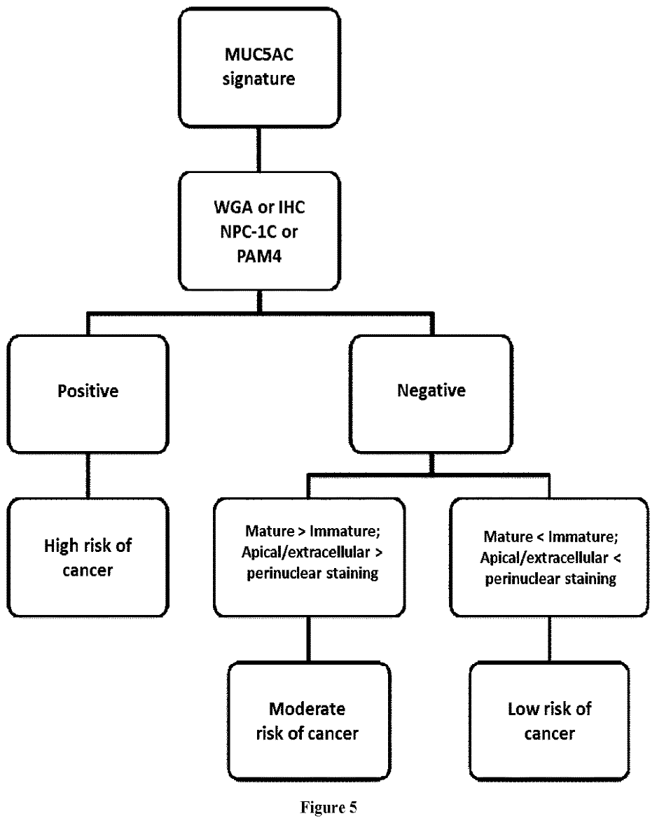

In patients with suspected PDA (IPMN or other cystic lesion in pancreas), MUC5AC signature can be used to diagnose PDA (FIG. 5). To improve the diagnostic sensitivity and specificity, a comprehensive integrative testing model is provided herein that includes tissue or fluid or cytology testing in the available patients and combine it with blood biomarkers as described in FIG. 3.

### Rationale for Individual Components in the Diagnostic Approach

Somatic mutations for liquid biopsy and cytology. Sixty-one percent (613/1009) of patients had at least one somatic alteration detected (from their panel of mutations) in cfDNA based on Comprehensive genomic profiling (CGP) (George, B., et al., Comprehensive genomic profiling (CGP) utilizing cell-free DNA (cfDNA) in patients (pts) with pancreatic ductal adenocarcinoma (PDAC). Journal of Clinical Oncology, 2021. 39(3_suppl): p. 421-421). TP53 was the most frequently altered mutation (55%), followed by KRAS (40%) and CDK2NA (6.5%). Other mutations that made the top 10 list include ATM (2.3%), PIK3CA (2.2%), PTEN (1.4%), TERT (1.4%), NF1 (1.4%), JAK2 (1.3%), and GNAS (1.1%). The frequency of the last 5 mutations in this list was under 1.5%. The frequency of the altered mutations in the blood (cfDNA) does correlate and that from the tissues as in the study published by the same group with the available data from 81 patients. KRAS, TP53, and CDK2NA were the top 3 mutated genes in both tissue and blood. In smaller studies prevalence of TP53 and KRAS is as high as 78% each.

Methylation markers in blood. This can be non-specific methylation as measured by 5-methylcytosine (5mC) and 5-hydroxylmethylcytosine (5hmC) or methylation patterns in the specific promoters (hypo or hypermethylation) as illustrated in Table 1. The list is prepared based on extensive literature search of the blood based methylation markers and combining them.

MUC5AC isoforms in blood. There is evidence that MUC5AC isoforms can be detected in the PDA patients (in serum and extra vesicles) and when it is combined with CA 19-9, the sensitivity and specificity of detection improves. Immunogenic glycoforms can also be detected in the blood of PDA patients.

MUC5AC signature in the tissue. As the detection of NPC-1C has high sensitivity, it increases the chances of diagnosing PDA. Depending on the agent used, it can be followed up by testing for mature and immature MUC5AC as discussed in the MUC5AC signature above. When mature (45M1) and immature (CLH2) were used to stain a tissue micro array (TMA) with 174 tissues including 70 adenocarcinomas and 104 non-cancer diseases (normal tissue, normal adjacent, islet cell tumor, pancreatitis). The sensitivity/specificity, respectively to diagnose pancreatic adenocarcinoma are noted below.
Mature alone: 36%/100%
Mature plus immature: 63%/92%
Immature alone: 42%/96%

Another TMA based study with 423 tumors showed 71% positivity with MSVA-109M (it has strong cytoplasmic staining, presumably immature) (Dwertmann Rico, S., et al., Mucin 5AC expression is common but unrelated to tumor progression in pancreatic adenocarcinoma. International Journal of Immunopathology and Pharmacology, 2022. 36: p. 039463202211065).

### MUC5AC Signature in Screening High-Risk Patients

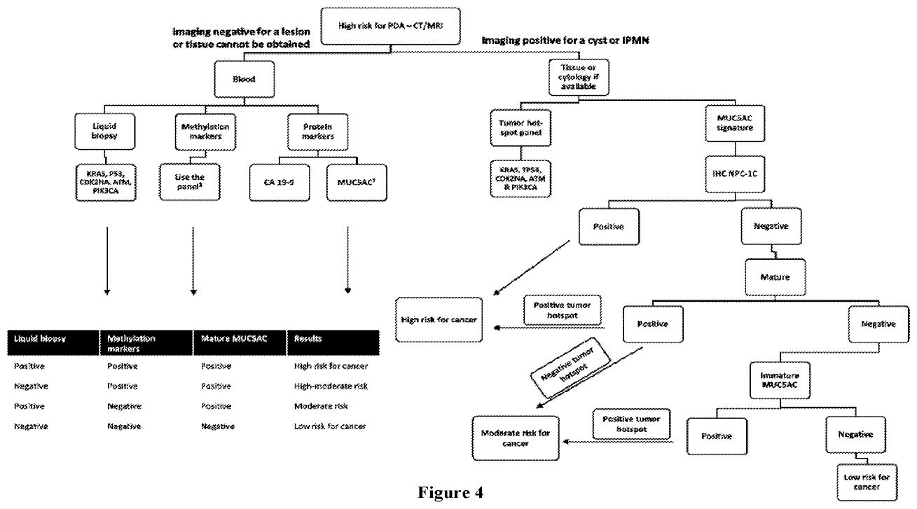

In high-risk individuals, addition of MUC5AC signature testing and blood markers as illustrated in the FIG. 4 below also can increase the sensitivity and specificity of screening for PDA.

### MUC5AC Signature for Predicting the Tumor Response.

### MUC5AC Signature in Tumor Tissue

The mature MUC5AC's role in the pancreatic cancer cell—viability, anchorage-independent growth, motility, adhesion to the extracellular matrix angiogenesis, apoptosis, and sensitivity to Gemcitabine was proved (Krishn, S. R., Secretory Mucin MUC5AC in Gastrointestinal Malignancies. Theses & Dissertations, 2016: p. 110). It was also shown that MUC5AC detected by CLH2 (immature) and 45M1 (mature) mabs are differently localized (Ho, J. J., et al., Secretion of MUC5AC mucin from pancreatic cancer cells in response to forskolin and VIP. Biochem Biophys Res Commun, 2002. 294(3): p. 680-6). NPC-1C-specific (immunogenic) MUC5AC has a distribution similar to mature MUC5AC. It can therefore be concluded that there are differences in chemosensitivities MUC5AC^{negative} MUC5AC^{low to moderate} and MUC5AC^{high} pancreatic cell lines (PCLs).

MUC5AC is expressed only in abnormal pancreatic tissues, but its clinical significance is ambiguous as the retrospective studies done so far were done using CLH2 mab that recognizes only immature MUC5AC. Mature MUC5AC (45M1 reactive) is present in apical and extracellular regions, and its secretion is influenced by the growth factors (forskolin and VIP) that have a proven role in malignant pancreatic cell growth.

The MUC5AC^{high} PCLs are more sensitive to 5FU, Cisplatin, Irinotecan, and resistant to Gem and Oxaliplatin than MUC5AC^{negative} PCLs. MUC5AC^{high} are more sensitive to 5FU, oxaliplatin and cisplatin, and resistant to Gem than MUC5AC^{low to moderate} PCLs. Thus, adding localization and other isoform data to the level of mature MUC5AC expression gives a better predictive signal in PDA patients. Analysis of patients from The Cancer Genome Atlas (TCGA) and Genotype-Tissue Expression (GTEx) data sets, MUC5AC expression was shown to be a poor prognostic marker even in gemcitabine treated patients.

### Test Design

Immunohistochemistry. The immunohistochemistry for MUC5AC using antibody clones CLH2 and 45M1, are performed at Clinical Immunohistochemical Laboratory, Department of Pathology, The Ohio State University Wexner Medical Center. The assay methodology development, optimization, and validation mirrors any new clinical immunohistochemical assay development in the laboratory. Commercially available MUC5AC mouse monoclonal antibody clones CLH2 (Novacastra, UK) and 45M1 (ThermoFisher, USA) are used herein. Briefly, the assay is performed on automated Stainer's (Leica Bond or Dako) in the laboratory. Appropriate positive (e.g., normal stomach) and negative tissue (e.g. normal pancreas) controls are used to optimize and validate the assay. The best pretreatment and antibody detection systems are determined using the manufacturer's antibody datasheet, and titration of at least three antibody dilutions are performed. If there is no recommendation from the manufacturer, the antibody is tested by trying the different lab pretreatment and detection system methods in order to determine the best method. Antibody dilutions may need to be optimized, depending on the results of initial testing. A pathologist reviews the slides from the validation run to determine the optimal methodology.

Assessment of MUC5AC immunohistochemical staining. The immunohistochemical-labelled whole tumor section slides is reviewed by a pathologist with expertise in pancreaticobiliary pathology. The slides are reviewed for the expression patterns (Cytoplasmic vs Membranous) and scored for the extent (% of tumor stain) and intensity of staining (Negative 0, mild 1+, moderate 2+ and strong +3). A semiquantitative approach of H-score is assigned to tumor samples for statistical analysis. The H-score is calculated by multiplying the percentage of the tumor staining and the predominant staining intensity with scores ranging from 0 to 300. Tumor with scores <10 are considered as negative for expression. Tumors with scores in the range of 10-300 are regarded as positive for individual MUC5AC expression.

PDA tumor samples with moderate/strong expression of mature MUC5AC and less immature MUC5AC exhibit poor outcomes. In addition, PDA samples expressing higher mature MUC5AC are associated with patients who had a better treatment response to 5FU based therapy compared to a gemcitabine-based therapy. It has been contemplated that some biopsy specimens may not have enough tissue to do immunohistochemistry (IHC), especially in advanced-stage cancers.

### MUC5AC Signature in Blood

There is evidence that MUC5AC isoforms can be detected in the serum of patients with PDA, however, relative isotype abundance was never measured, nor has expression been correlated to treatment response.

Test design. To characterize the expression pattern of MUC5AC glycoforms in PDA patients, the same specific mAbs (CLH2 and 45M1) are utilized to evaluate the serum samples of treatment naïve PDA using a highly sensitive, novel sandwich ELISA-assay. Batched serum of PDA patients are captured and detected using the glycoform-specific mabs as above. To ensure the consistency and reproducibility of the assay, a one-time prepared lysate of A549 cells, which innately express a high-level of MUC5AC, are used. Commercial serum from a normal individual (no PDA) are used as an internal negative control. Reciprocal titers are determined, and the levels of MUC5AC are reported as nanogram per ml of total protein in the A549 cells.

The sera from patients with PDA with high levels of mature MUC5AC at diagnosis respond better to 5-FU based therapy than gemcitabine-based therapy.

### Example 2: Summary Report

Mucin 5AC (MUC5AC) is a glycoprotein identified in pancreatic ductal adenocarcinoma (PDA), with strong preclinical evidence showing its impact on treatment response. The distribution of two major glycoforms of MUC5AC, heavily glycosylated (mature) and less-glycosylated (immature) forms identified and studied by 45M1 and CLH2 monoclonal antibodies, respectively, in resected PDAs. The serum MUC5AC expression was also studied in these patients, using commercially available ELISA kits to predict the treatment response.

One hundred resected PDA specimens and serum samples were collected. 43 patients had neoadjuvant therapy (NAT), and 57 had upfront surgery (UpS). The cellular expression of MUC5AC was studied in the resected formalin-fixed paraffin-embedded tissue through immunohistochemistry using 45M1 and CLH2 antibodies. The expression levels in the tissue were estimated using H-score intensity (0-3)X % of tumor cells with expression with results ranging from 0-300. The presence or absence of extracellular (EC) expression was also studied. Below is a summary of some significant findings.
CLH2-MUC5AC was identified in the perinuclear region or cytoplasm, while 45M1-MUC5AC was identified in apical and EC regions. The expression (intensity and % distribution) of CLH2 and apical 45M1 were similar in all tumor cells.
All treatment naïve patients (UpS) expressed both glycoforms compared to 90% in NAT group in tumor tissue. EC-45M1-detection rate was 82% in UpS group vs 58% in NAT group.
CLH2 expression and 45M1 EC expression were significantly lower in the NAT group without metastasis when compared to the UpS group (H-score 122 vs. 162, p=0.02) and (EC-detection rates 58% vs. 82%, p=0.007). 45M1 expression showed an encouraging trend with lower expression in NAT when compared to UpS group (H-score 129 vs. 164, p=0.06).
If the NAT group is broken down based on pathological response identified in the resected sample as objective response (OR), including both near complete response (nCR) and partial response (PR) and no response (NR=minimal or no response), and compare them with UpS group, the differences in CLH2 (OR vs. NR vs. UpS=H-scores 104 vs. 152 vs. 163, p=0.01) and 45M1 EC-detection rates (56% vs. 63% vs. 82%, p=0,02) were significant. At the same time, 45M1 expression showed a trend towards being significant (H-scores113 vs. 154 vs. 164. p=0.06).
   On head-to-head comparison, OR vs. UpS were significantly different (p<0.05) for expression of CLH2, and 45M1, and detection of EC-45M1
   There was a reasonable number of patients with FOLFIRINOX (combination of 5FU, oxaliplatin, and irinotecan) NAT(N=36), and the expression in this group was similar to the entire NAT group (N=43).
      In 5 patients who had gemcitabine and nab-paclitaxel (Gem-NP) NAT, the intracellular MUC5AC expression for both CLH2 and 45M1 were lower in NR group vs. OR group (45 M1 H-score 80 vs. 140 and CLH2 H-score 80 vs 96). Interestingly, the OR group had higher EC-45M1 detection rates than the NR group.
On univariate logistic regression, treatment and response (OR vs. NR vs. UpS) showed a significant impact on 45M1 and CLH2 expression levels (Mlp=0.03 and p=0.01, respectively) and EC-45M1 detection rates (p=0.008). NAT, tumor size (<2 cm vs. >2 cm) and associated premalignant lesions (IPMN or PanIN) were other factors that had an impact on MUC5AC expression levels.
In NAT group,
   On univariate analysis (UVA), 45M1 and CLH2 expression levels did not have any significant impact on progression-free survival (PFS) and overall survival (OS).
   On multivariate analysis (MVA),
      CLH1 and 45M1 expression were significant for PFS (hazard ratio (HR) of 0.959 (p=0.01) and 1.040 (p=0.03) for 45M1 and CLH2, respectively) and OS (HR of 0.9 (p=0.03) and 1.040 (p=0.03) for 45M1 and CLH2, respectively).
      EC-detection had a p-value of 0.07 but impressive HR (0.30 for EC-negative) for PFS.
A similar analysis showed no impact of MUC5AC expression in UpS for PFS or OS
There were 9 metastatic tissues to compare the MUC5AC expression with primary tumors. Five of them were heavily treated with systemic therapy. CLH2 was significantly higher in metastatic sites (196 vs. 145 (p=0.04)) while 45M1 (199 vs. 149, p=0.05)) and EC-45M1 detection rates (100 vs. 72, p=0.06) had a trend towards significance.
   Serum samples for the patients were collected at different time points of the management. This allowed for studying the relationship between serum MUC5AC with the known pancreatic cancer tumor marker CA 19-9 and MUC5AC's role in predicting treatment response. Human serum MUC5AC levels were analyzed using the Human MUC5AC ELISA Kit (Catalog number NBP2-76703, Novus Biologicals, Centennial, CO). 45M1 and CLH2 could not be detected in the serum samples, but the commercially available kits allowed detection and measuring of MUC5AC. The results are as follows.
In treatment naïve (N=11—post-diagnosis, before systemic therapy, and before surgery) patients, the mean serum MUC5AC and CA 19-9 were 3.6 and 955 ng/mL. There was a positive correlation between them.
Twenty-three serum samples of patients were on NAT (before surgery, 19-FOFLIRINOX, 1-FOLFOX, and 3—Gem-NP). The serum was collected within a week to 25 weeks after the first dose of chemotherapy (median of 5 weeks). The mean MUC5AC levels (in ng/mL) was 1.82, median 0.7 (0.4 to 8.3).
   Patients with PR had significantly higher mean serum MUC5AC than those with nCR and NR (nCR vs. PR vs. NR, 0.43 vs. 2.9 vs. 1.2, p=0.01). The CA 19-9 levels followed the same trend (nCR vs. PR vs. NR=129 vs. 452 vs. 958, p>0.05). The patients who had OR (nCR+PR) had higher MUC5AC levels compared to NR (2.4 vs. 1.2, p>0.05) but lower CA 19-9 level (393 vs. 958, p>0.05).
      When the group was divided into two based on cut-offs such as median (0.7 ng/ml), mean (1.82 ng/ml), and 1 ng/ml (it was the mean for patients with NR in the FOLFIRINOX group), lower MUC5AC groups had higher levels fraction of nCR and NR than PR, and vice versa. This can explain larger tumors in these groups. Alternatively, other key determinants of outcomes, such as residual disease and margin-positivity, were significantly (p<0.05) lower in this group, explaining significantly lower PFS for a cut-off of 1.82 (9 vs. 3 months, p=0.04). It should be noted that CA 19-9 levels were very high but not statistically significant in lower MUC5AC groups. One key takeaway from this is that PFS is not only driven by the pathological response in NAT group. Clinically, this may translate into guiding the physicians about the timing of surgery while on systemic therapy. Waiting till MUC5AC and CA 19-9 dropping could be an ideal time.
      Similar findings were noted in FOLFIRINOX (N=19) group (nCR vs.PR vs. NR=0.43 vs. 3.2 vs. 0.9, p=0.0013; OR vs. NR, 2.64 vs. 0.9, p=0.05).
There were 17 patients within 8 weeks of surgery, 9 within 5 weeks, and 8 from 5-8 weeks. The mean MUC5AC was lower than the ones noted in the pretreatment/presurgery population (1.2 ng/mL). Among them, higher MUC5AC (>0.5 to 1 ng/mL) tend to have a higher rate of recurrence and low PFS (in our population, p was >0.05).
There were very few patients with serum available during surveillance or while on palliative systemic therapy to draw any concrete conclusions.
   Detectable MUC5AC, irrespective of normal or undetectable CA 19-9, could be an indication of disease progression (PD) while on surveillance or adjuvant therapy (AT), as seen in 6 patients of the study population (2 were on surveillance and 4 were post-AT).
   Low/rising MUC5AC with abnormal CA 19-9 could indicate early treatment resistance in patients on palliative systemic therapy even if the scans do not show clear-cut PD.
   There was not enough data to propose treatment selection by serum MUC5AC level, but higher MUC5AC levels were noted in patients who recurred while on Gem-only or Gem-based AT therapy.

### Conclusion

Tissue MUC5AC expression signature that includes CLH2 and MUC5AC expression levels (H-scores) and the presence or absence of EC-45M1 gives insight into treatment response in patients who receive NAT and then proceed for curative resection. This study provides evidence for a commonly used NAT regimen, FOLFIRINOX-Orin tissues is often associated with lower MUC5AC levels and EC-45M1 detection rates. If the tissue MUC5AC levels do not drop but EC-45M1 detection is undetectable, patients may not metastasize while on NAT and proceed to surgery even if they do not have an appreciable response.
EC-45M1 detection is an independent risk factor with an encouraging trend (p=0.07) toward significance for worsening PFS in patients receiving NAT. The intracellular MUC5AC had a significant association with PFS and OS.
Serum MUC5AC in treatment naïve patients could indicate tumor burden as it could be the EC-MUC5AC from the tumors (primary or metastatic). Detectable or rising MUC5AC within 8 weeks post-surgery or AT or while on AT could indicate recurrence, especially if there is an associated increase in CA 19-9.
While on treatment, the MUC5AC, in conjunction with CA 19-9, could help detect treatment resistance.
   While on NAT, 2-4 weeks after initiation of chemotherapy, the relationship between MUC5AC, CA 19-9, and treatment response noted in the resected sample is summarized as follows.

### Example 3: Summary of Relationship Between MUC5AC and CA19-9

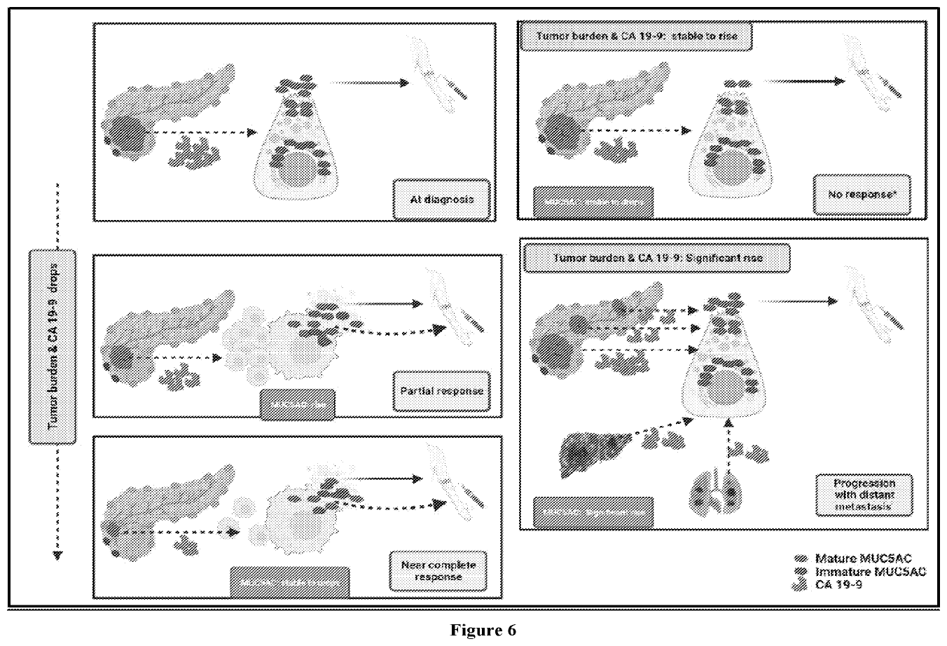

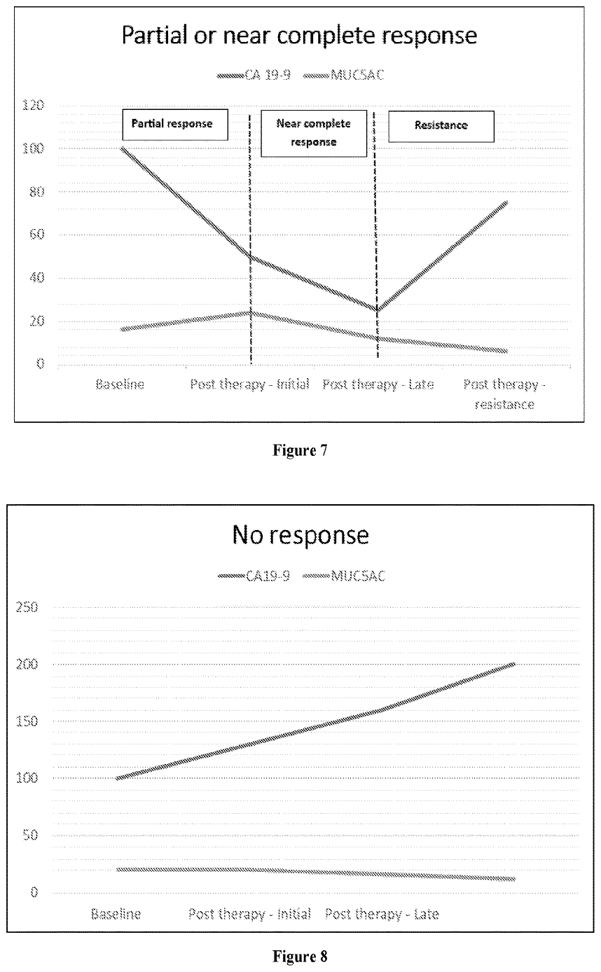

FIGS. 6 and 7 summarize the relationship between MUC5AC and CA19-9 with tumor burden and treatment response.
Before any interventions (such as at diagnosis before systemic therapy or surgery), post-curative resections (5-8 weeks postoperative period), and while on surveillance, the serum MUC5AC mainly represents the EC-MUC5AC.
After initiating NAT,
   If patients respond to the systemic therapy, tumor cells start dying, and intracellular MUC5AC is released into the bloodstream. This accounts for a rise in serum MUC5AC and a drop in CA 19-9. Surgery at this point could show PR in the resected specimen.
      If therapy is continued beyond this, the fraction of remaining tumor cells is low. Hence, the MUC5AC released into the bloodstream is low, accounting for its stable to low levels accompanied by low CA 19-9. Surgery at this point will show nCR or CR.
      If the systemic therapy does not have a considerable effect on the size of the tumor but is enough to prevent its progression, the EC-MUC5AC will drop, but the intracellular MUC5AC will remain intact and is not released into the bloodstream (as the cells are not dying). The MUC5AC will be low or stable from diagnosis, and CA 19-9 levels could be stable to high.
   If the patient is not responding and is progressing with distant metastasis, the tumor burden increases. Hence, the EC-MUC5AC and CA 19-9 continue to rise.
This supports FOLFIRINOX treatment. The numbers for Gem-NP or FOLFOX are low.
   It has been contemplated that shedding mature MUC5AC is a resistance mechanism specifically for Gem or NP or high MUC5AC, facilitating response to them.

### Example 4: Clinical and Translational Strategies

### Clinical:

A trial is designed that involves a prospective collection of blood samples in patients treated with FOLFIRINOX, stratified by NAT (expected to have curative resection), and palliative therapy for advanced PDA. A composite index with serum CA 19-9 and serum MUC5AC levels/changes is used to predict treatment response/resistance and tumor burden.
   Addition of cell-free DNA testing for frequently identified mutations (KRAS and TP53) and epigenetic markers is also explored to develop a multiomic test to identify early treatment resistance.
Next, an interventional study is developed to help in treatment selection and early resistance monitoring.

### Translational:

Preclinical studies using cell lines, organoids, or animal models to understand:
   Triggers for MUC5AC production in pancreatic adenocarcinoma.
   MUC5AC role in distant metastasis, local invasion, and treatment resistance
   Effect of MUC5AC blockade using known molecules such as azithromycin, phosphodiesterase type 4 (PDE4) inhibitors, 6-mercaptopurine, on signaling pathways such as MAPK, Notch and EGFR.

### Example 5: Serum Analysis of MUC5AC

### Serum MUC5AC at Diagnosis.

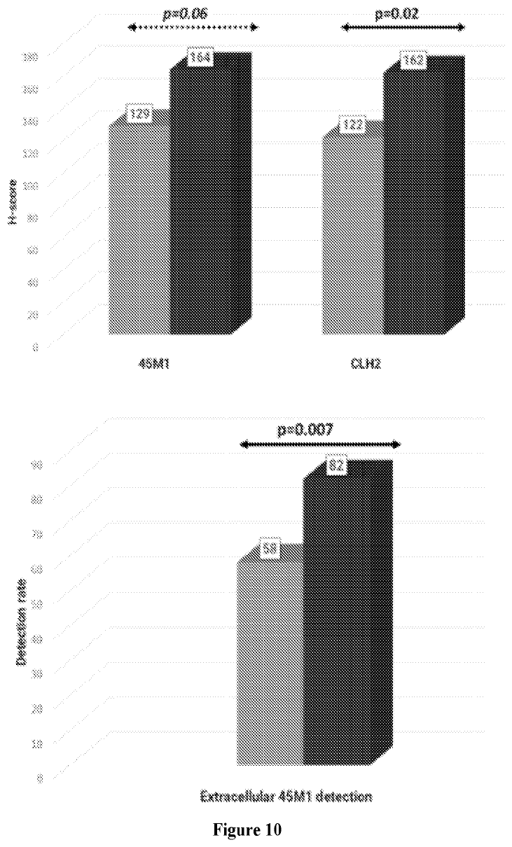

There were 11 (9/11 from the neoadjuvant therapy (NAT)-group and 2/11 from upfront surgery (UpS)) for analysis. The mean MUC5AC level (in ng/mL) is 3.6, with a median of 0.8 (0.41 to 26.2). In NAT-group, 7/11 had FOLFIRINOX, one had FOLFOX, and one had Gem-NP.
Comparison with CA 19-9:
There was CA 19-9 from 9/11 on the same day, 1 within a week, and another within 4 weeks of MUC5AC levels. The mean CA 19-9 level (in ng/mL) was 955, with a median of 356 (22.75 to 3701). Zero (0) was used for analysis throughout in patients with undetectable CA 19-9 (<15 ng/mL). The two biomarkers had a statistically significant (p=0.0078) positive correlation (FIG. 10).
Correlation with Tissue MUCAC Expression:
There were only two patients with UpS to study this in this population. The serum MUC5AC (in ng/mL) levels of these patients were 0.6 (CA 19−9=461 ng/mL) and 0.4 (CA 19−9=246 ng/mL). The one with lower serum MUC5AC but high CA 19-9 had lower tissue-MUC5AC distribution and H-scores for both glycoforms (CLH2 and 45M1, H-score, 90 vs. 150, % distribution, 30% vs. 50%) than the other. Both were extracellular (EC)-45M1-positive and had R0, T2, margin-negative, and PanIN-associated tumors.

### Conclusions

In treatment naïve PDA (at diagnosis or before surgery and systemic therapy), serum MUC5AC levels directly correlate with CA 19-9 levels, and the mean serum MUC5AC levels is around 3.6 ng/mL with a median of 0.8 ng/mL.
In patients with UpS, serum MUC5AC predicts tissue MUC5AC expression levels.

### Serum MUC5AC as a Predictor for Pathological Treatment Response.

There was an analysis of patients with serum MUC5AC levels available post-NAT before and before surgery to understand if serum MuC5AC levels help predict treatment response. The pathological treatment response was used as a main indicator for the response evaluation. For practical purposes, patients with extensive tumor with no evident tumor regression or 3 were designated as no-response (NR) group. Patients with residual tumor with evidence of regression or 2 and near complete response or single cells or small group of cancer cells or 1, were considered as partial-response (PR) or near complete-response (nCR). Objective response (OR) group refers to patients with PR and nCR. Other pathological factors which are considered key for outcomes such as residual disease (R0—no residual disease and R1-2 with residual disease), margin-status, tumor size (T1 (≤2 cm) vs. T2-3 (>2 cm)), and node positivity (N0 vs. N1-2), perineural invasion (PNI), and lymph vascular invasion (LVI) were also studied. The correlation between tissue (45M1 and CLH2) and serum MUC5AC levels was analyzed as the former correlated with treatment response in NAT-group.

There were 23 patients in this group to analyze. Nineteen (19) had FOLFIRINOX, 3 had gemcitabine and nab-paclitaxel (Gem-NP), and one had FOLFOX. The serum was collected within the week of first dose up to 25 weeks (median of 5 weeks).

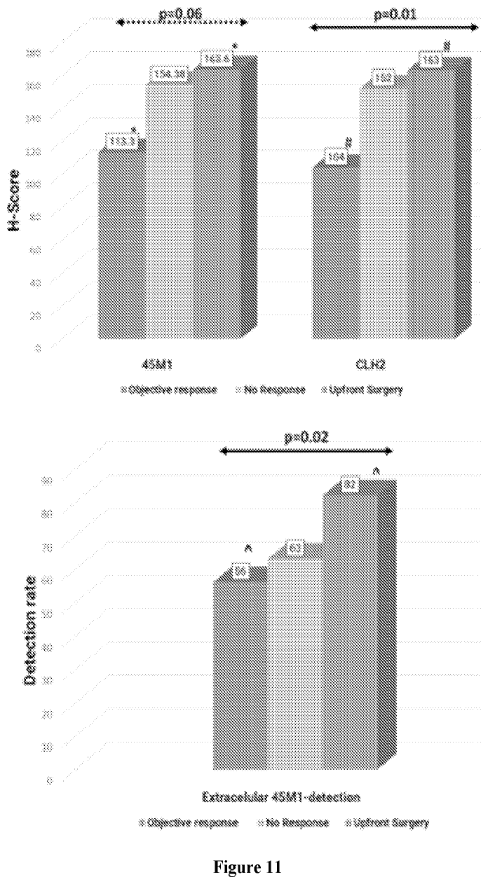

The mean MUC5AC levels (in ng/mL) was 1.82, with a median 0.7 (0.4 to 8.3), quartiles 0.43 and 2.09 for 25 and 75 percentiles, respectively. Mean CA 19-9 levels (in ng/mL) were 698, with a median of 207 (0-5874). Three (3) patients had undetectable CA 19-9 (<15) on the day their MUC5AC was measured (2 with FOLFIRINOX and one with Gem-NP). The correlation between CA 19-9 and MUC5AC was not statistically significant, but there was a negative correlation (as opposed to a positive correlation before surgery) in all 23 patients, FOLFIRINOX (N=19) or 5FU-based (FOLFOX+FOLFRINOX). The FOLFIRNOX correlation is reported in FIG. 11. There was a significant positive correlation between serum MUC5AC and tissue 45M1 (p=0.04), CLH2 (p=0.07), and EC-45M1-detection (0.05). The combination of EC-45M1-H-score also correlated with serum MUC5AC levels (p=0.01).

For analysis of serum MUC5AC levels post-NAT, the serum MUC5AC levels were compared during NAT with the pathological treatment response observed in the resected specimen.

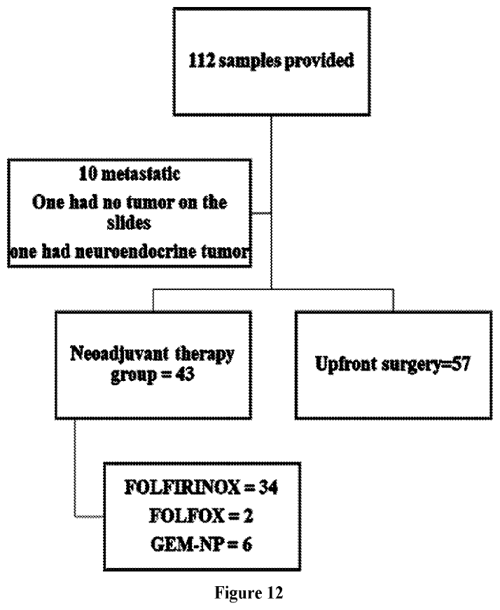

MUC5AC levels (in ng/mL) were significantly different (by logistic regression) among the patients who had nCR vs. PR vs. NR (0.43 vs. 2.8 vs. 1.2, p=0.01) in this population (FIG. 12). Among these groups, CA 19-9 levels (in ng/mL) were proportional to the response noted in the resected sample, but the difference was not statistically significant (nCR vs. PR vs. NR=129 vs. 452 vs. 958, p>0.05). When we combined nCR and PR groups to OR and compared it with NR group, the serum MUC5AC levels are higher in OR than NR (2.4 vs. 1.2, p>0.05), and CA 19-9 levels are predictable (393 vs. 958, p>0.05).

The population was divided into two groups using three cut-offs for serum MUC5AC levels, i) median, 0.7 ng/mL, ii) mean, 1.82 ng/mL, and iii) 1 ng/mL (this threshold is explained in the following section). The details are in Table 3.

The theme is similar for all the thresholds. The lower MUC5AC group tends to have higher NR and nCR patients than PR. This explains the lower MUC5AC expression levels in the resected specimens (explained below) and higher serum CA 19-9. Interestingly, they also have a higher fraction of larger tumors (T2-3) but lower residual (R1-2) and margin-positive tumors. Other factors are mentioned in Table 3. As the cut-off threshold increases, the ability to distinguish OR vs. NR, nCR vs. PR vs. NR improves. Serum CA 19-9 levels were 5-10 times higher in NR than in nCR patients, which could help differentiate them. The progression-free survival PFS was significantly higher (9 vs. 3 months (p=0.04)) in the lower MUC5AC group.

A similar analysis was performed in the 19 patients who got FOFLIRINOX as it is the most preferred regimen in the clinical practice for the eligible patients. The median time for serum collected was 6 weeks (1-16) after therapy. The mean MUC5AC level (in ng/mL) was 1.74, with a median of 0.7 (0.43 to 8.3). There was a significant difference (p=0.0013) in MUC5AC levels (in ng/ml) among nCR (0.43), PR (3.2), and NR (0.9) groups (FIG. 13). The serum CA 19-9 levels were relative to the response noted (129 in nCR, 37 in PR, and 1060 in NR). The difference in MUC5AC levels between OR and NR had a trend towards significance (OR vs. NR, 2.64 vs. 0.9, p=0.05). The threshold of 1 ng/mL was taken as it was the mean level in NR-group receiving FOLFRIINOX.

This group was further divided using a similar strategy employed for cut-offs (Table 4). The median (0.7 ng/mL) and mean (1.82 ng/<mL) were used. This was also tested using the threshold of 1 ng/ml (rounded off the mean level noted in NR-group, 0.9). The observations were similar except tissue MUC5AC expression levels. The lower MUC5AC group had a higher fraction of NR patients, which could explain this. The PFS was again high for low MUC5AC group (9 vs. 2.5 m, 0.2). The trend was similar even for the threshold of 1.8 ng/mL.

Low MUC5AC and PFS in patients receiving NAT: A comprehensive examination was performed on the limited data to compare clinical aspects among the groups for various serum MUC5AC thresholds (in ng/mL), such as 0.43 and 2.09 along with the one noted above. Two patients with nCR and 2/12 with NR had 0.43 ng/mL. Even though the fraction of NR and larger tumors were higher in low MUC5AC groups, they had fewer patients with margin-negative and residual disease. This again proves that the biology of the tumor is important. Higher MUC5AC groups may have PR or NR, but they have other poor risk factors. CA 19-9 was higher in the mid-range MUC5AC (0.43 to 0.7 ng/mL).

There is indications that a patient with nCR will have PR first. If the tumor responds to therapy but a large amount of tumor is left (PR), the MUC5AC level in the serum rises, and the CA 19-9 level drops. If the tumor continues to respond and only a small amount of tumor is left, i.e., nCR, MUC5AC level drops, and CA 19-9 continue to drop (FIG. 8).

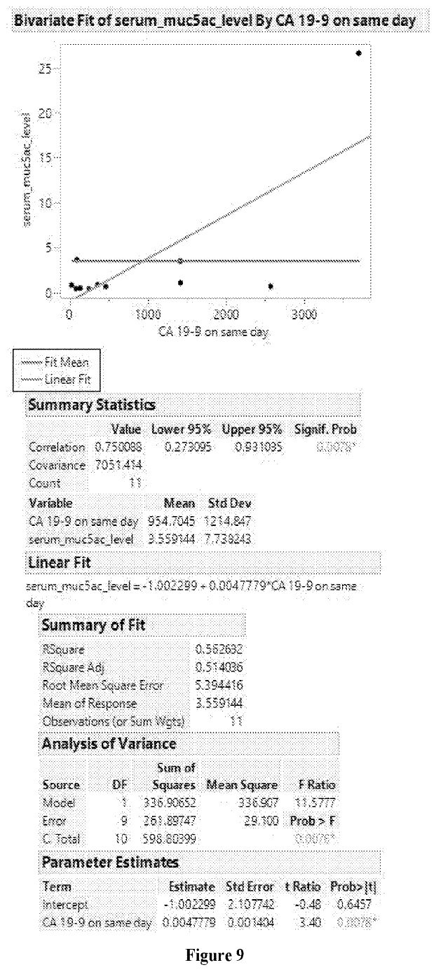

When the tumor develops resistance, the cells do not die; hence, there is less MUC5AC in the blood and high CA 19-9. In patients who do not produce CA 19-9, MUC5AC levels rise with response and plateau or are stable with no response. Thus, combining CA 19-9 and MUC5AC levels is an optimal route. Alternatively, in patients with NR, PDA cells do not die, less MUC5AC is detected in the blood, and they tend to produce higher amounts of CA 19-9 (FIG. 9).

Differentiating nCR and NR: The relationship between serum CA 19-9 and MUC5AC and the pathological response is summarized and noted in the resected sample. Rising CA 19-9 with low-stable MUC5AC could indicate NR (CA 19-9, NR vs. nCR, 1060 vs. 129, p=0.2). Low to stable MUC5AC with dropping CA 19-9 could indicate nCR. On the other hand, if CA 19-9 and MUC5AC rise, it could indicate disease progression.

The individual patients with nCR were studied. One had undetectable CA 19-9 and low MUC5AC (0.43 ng/ml) in the sample collected 21 weeks after the first dose of chemotherapy. CA 19-9 was always within the normal range in this patient. Conversely, MUC5AC was low (0.43 ng/mL), but CA 19-9 was 258 on the same day. It was collected 5 weeks after treatment. CA 19-9 dropped from 530 to 258 as the treatment continued indicating good response. In both cases, the sample can be taken after or close the PR phase (Table3).

In patients with NR in the FOLFIRNOX group, 3 had normal CA 19-9 after 4-15 weeks with >1 MUC5AC. All three had near-normal or undetectable CA 19-9 up to this point. The rest (N=7) had higher CA 19-9 and <1 ng/mL MUC5AC. The patient with Gem-NP who had NR also had higher CA 19-9 and very low MUC5AC (0.43 ng/mL) 8 weeks after therapy. Interestingly, even though the resected specimen had minimal CA 19-9 expression (CLH2/45M1 H-score was 10), they had NR. This patient also had other poor pathological factors (T3/node/PNI/node/PNI/LVI—positive but R0 and margin negative). Another patient with FOLFOX also had MUC5AC of 5.6 with CA 19-9 of 741, but as the treatment continued, his CA 19-9 continued to rise to more than 2000 in the next couple of months. The resected specimen had all poor prognostic indicators, large tumor (>5 cms), and high MUC5AC expression (CLH2/45M1 H-score was 270).

Finally, two patients with Gem-NP had PR. One had high MUC5AC (2 ng/mL) but undetectable CA 19-9. Other had low MUC5AC (0.6 ng/mL) but very high CA 19-9 (1711 ng/mL) at 25 W but had CRT after the blood was drawn, and the CA 19-9 3 weeks before surgery was 164, indicating the clear benefit of CRT. This patient's tissue MUC5AC was very low (CLH2/45M1 H-score was 30, EC-45M1-negative).

### Conclusion

Post-systemic therapy (any combination) MUC5AC and CA 19-9 can give an accurate understanding of treatment response before imaging and within 1-4 weeks.
   If MUC5AC levels are stable or dropping and CA 19-9 rises, it could indicate poor/no response.
   If MUC5AC levels are rising with a drop in CA 19-9, it could indicate a response. Over a period of time,
      if CA 19-9 continues to drop and MUC5AC is stable or trending further down, it could indicate nCR or stable disease.
      If CA 19-9 starts trending up with dropping/stable MUC5AC, it could indicate resistance.
In patients treated with FOLFIRINOX, if the MUC5AC level drop to <2 or trends that way after 2-3 doses, they tend to have poor response and nCR if associated with rising and dropping CA 19-9, respectively, but if they proceed with resection, they could have smaller tumors with R0 and margin-negative disease. Alternatively, if the MUC5AC levels are >1 ng/mL, they will have more OR or PR but are at a high risk of R1-2 and margin-positive disease, which worsens their outcome. The ideal cut-off was unclear from our study; we expect it to be from 1 to 2.
Although there are very limited patients treated with Gem-NP or FOLFOX, but the overall treatment-related trend seems to be the same.

### Serum MUC5AC as a Predictor for Recurrence Post-Surgery.

Early MUC5AC levels as a predictor for recurrence: MUC5AC levels were obtained on 9 patients within 5 weeks post-surgery. The mean serum MUC5AC levels (in ng/mL) were 1.3 with a median of 0.83 (0.43 to 3.325). Mean CA 19-9 (in ng/mL) was 15 with a median of 0 (0-87). Six patients had undetectable (<15) CA 19-9, two had normal (25 and 26), and only one had higher CA 19-9 (87).

The patients were divided into two groups with the median as cut-off (<0.83 (N=5) vs. >0.83 (N=4)) to compare the characteristics. In the lower serum MUC5AC group, one had 3 doses of FOLFIRINOX NAT; one had CRT, one had Gem-only, and the other two had Gem/cap. In the higher MUC5AC group, 3/4 had Gem-only as adjuvant therapy (AT) while the others got Gem/capecitabine. Even though it was not statistically significant, lower MUC5AC group had lower tissue-MUC5AC expression (CLH2/45M1 H-score was 132 vs. 187), higher median PFS (58 vs. 19 months) and less risk of recurrence (40% (2/5) vs. 75% (3/4)). In this group, one patient who had Gem-only had both local and distant (liver) in 15 months. The CA 19-9 was done on the same day as MUC5AC was elevated (87). The other had undetectable CA 19-9 and 0.7 MUC5AC, but R1 disease had CRT with 5FU sandwiched between 2 cycles of Gem. This patient had a distant (lung) 5 years after surgery. The three recurrences in the higher MUC5AC group had undetectable or normal same-day CA 19-9.

Later (5-8 weeks) MUC5AC levels as a predictor for recurrence: 5-8 weeks. The mean MUC5AC levels were 1.27, with a median of 0.76 (0.4 to 3.42). The mean CA 19-9 was 166, with a median of 28.5 (0-619). The population was divided with a MUC5AC level of 0.76 (<0.76 (N=4) vs. >0.76 (N=4)). The trend is the same as with 1-5 weeks of analysis (data not shown here). The higher MUC5AC group has 100% recurrence despite having normal CA 19-9 with low median PFS (12.5 vs. 35 months, p=0.2). Low MUC5AC group with high CA 19-9 are at the risk of recurrence.

A similar trend was noted when the thresholds were moved to 0.5 or 1 ng/mL MUC5AC. Most of the patients got Gem-only or Gem-based AT. It is hard to conclude, but Gem-based therapy may not be ideal if MUC5AC is detectable.

If both the patient populations (post-surgery up to 8 weeks irrespective of therapy, N=17) are combined. A similar trend was noted: higher MUC5AC (>1 ng/ml), even with lower/undetectable CA 19-9, are at higher risk (88% vs. 60%) of recurrence.

### Conclusion

High MUC5AC (threshold between 0.5 to 1) within 8 weeks of surgery; there is a high risk of recurrence despite having normal CA 19-9. Gem-based therapy may not be useful.

### Serum MUC5AC as a Predictor of Treatment Resistance in Advanced PDA.

There were 10 patients with serum MUC5AC levels available at the time of documented recurrence (N=7) or progression (N=3). The population was used to see if MUC5AC can be an indicator of treatment response/resistance in advanced PDA treated with systemic therapy. For this group, the mean CA 19-9 was 4 with a median of 1.7 (0.5 to 24), and CA 19-9 was 123 with a median of 45 (0 to 109,233). For all practical purposes, the patients were divided into three major groups (Table 5): patients who progressed while on surveillance, patients who progressed on adjuvant therapy (PoAT), and patients who progressed to the second line of systemic therapy (PoSL).

Key takeaways are that detectable MUC5AC is concerning for recurrence in patients on surveillance. CA 19-9 is unreliable while patients are on AT, and trending MUC5AC helps. For PoSL, combining CA 19-9 and MUC5AC levels predict the response and resistance. For PoSL 1 in Table 3, there was no documented PD on the scans even if the CA 19-9 was rising, Irrespective of chemotherapy (NAT or AT), patients with higher serum MUC5AC before and after surgery had a risk of recurrence (Table 6). Similarly, patients with serum available to measure MUC5AC post-recurrence, had a mean of 6.4 ng/mL.

In patients from UpS group, after surgery and before recurrence, and on Gem-only or Gem-based therapy had higher MUC5AC level (Table 7) than the no-recurrence group (Table 6).

The goal here was to see if MUC5AC levels help in detecting early treatment resistance, which is key for patients on NAT, and explore treatment selection as preclinical evidence indicates MUC5AC imparts Gem-resistance. The NAT-group was used for analysis which has 9 patients for this analysis.

The FOLFIRNOX-group (N=7) was the first focus. The mean MUC5AC and CA 19-9 levels (in ng/mL) were 1.12 with a median of 0.8 (0.43-3.5) and 857 with a median of 356 (22.75 to 2576), respectively. The patients were divided into two groups based on a median MUC5AC level for this group, 0.8 as cut-off (≤0.8 (N=4) vs. >0.8 (N=3). The results were in line with the ones discussed above with the post-FOLFIIRNOX group (Table 4). The group with lower baseline MUC5AC had significantly lower R1-2 (25% vs. 100%, p=0.047) and margin-positive disease (25% vs. 100%, p=0.04) tumors. Even though statistically not significant (p>0.05), they had higher MUC5AC expression levels (45M1 (H-score/% distribution, 170/57vs. 112/37) & CLH2 (160/57 vs. 92/37)) and larger tumors (T2-3 rate, 75% vs. 66%) in resected samples. That can explain this group's higher NR fraction (25% vs. 0%). These patients were heavily treated for 4-5 months and 3 even had neoadjuvant chemoradiation (CRT). It should be noted that one patient with low baseline CA 19-9 and MUC5AC got CRT, while 2 with higher CA 19-9 and MUC5AC had CRT.

One patient had CA 19-9 of 3701 with MUC5AC of 26.2, had 3 months of Gem-NP followed (no appreciable response noted on imaging) by CRT with capecitabine that resulted in R2 pathological response with R1, T2, N-positive, margin-positive disease. A patient who got FOLFOX as NAT had a serum MUC5AC level of 3.6 ng/mL (CA 19-9 was 93 ng/mL) approximately 12 weeks before the first dose. A similar trend was observed in the entire NAT-population in this analysis (N=9), with 0.807 (N=4 for <0.8) as a cut-off.

### Conclusion

Trending MUC5AC while on adjuvant therapy or surveillance identifies early recurrence or treatment resistance when combined with CA 19-9.
Higher MUC5AC levels were noted in patients who recurred while on Gem-only or Gem-based therapy

### Serum MUC5AC in Metastatic Patients.

There were 10 metastatic patients in the study. One had microscopic liver metastasis. The biopsy resulted after the primary resection. The serum sample available was collected 24 weeks post-surgery while on systemic therapy. The MUC5AC was 0.5 ng/mL, and CA 19-9 on the same day was 90/mL. Serum MUC5AC levels were obtained on 8 patients before 1-5 weeks of surgery. This threshold was picked because NAT is usually stopped at this time period. There is an overlap of patients among the groups analyzed above including 2 patients with UpS. If the patients are divided into two group with a threshold of 1 ng/mL, ≤1 (N=5) vs >1 (N=3), Lower MUC5AC level group had significantly, higher CA 19-9 (194 vs. 20 ng/nL, p=0.001, FIG. 12). Even though it is not statistically significant, they tend to have higher CLH2 and 45M1 expression and R0, N0, margin-positive, and larger tumors. For NAT-group, one patient in this group had NR while 2/3 in the other group had NR.
>0.5 vs. <0.5, 5 vs. 3
>0.5, 100% risk of recurrence (distant mets). All had normal CA 19-9
   Two had NAT with FOLFIRINOX and GA, limited post-perative therapy (one had 2 doses of FOFLIRIONX<other was enrolled in vaccine trial)
   both had NR, one had Gem-Np, no NAT,
<0.5,
   both distant mets in patient with high CA 19-9 (>500), gem and Gem/cap—ful therapy was given
   Distant met only, low CA 19-9 (44) but had R1 disease, got CRT with 5FU sandwitched between Gen
   Rec at surgical site, patient had Gem-NP, and recurrence was after 54 Months.
   Did not recur, had low CA 19-9, gem-only for 6m no concerning path features
For >1 vs. ≤1 (3 vs. 5)

### Conclusion,

>0.5, 4-8 weeks high risk of recurrence. Need longer courses of AT. Gem may not be enough.
   Within all 8 weeks (17)
>1 vs. ≤1,
   >1, high risk of mets, 88% distant mets, 1 no rec (≤1, 60% rec, )
   Low CA 19-9, 17 vs. 134, all of them have normal CA 19-9
   PFS 14 vs, 54 (p=0.2)
   If we remove three patients with NAT,
      >1, high risk of recurrence, 80% (vs 67%), low PFS
      Low CA 19-9
−5 to +6 for surgery (n=7)→increase to 10 weeks
Mean MUC5AC was 1.87, 18 (0.52 to 3.4). 3/7 had undetectable CA 19-9, 1 had norma, 32. Other had 54. One with 1.7 had 3292 and other with 2.9 had CA 19-8 of 10347.
one with R1 had undetectable and other with 1037
All >0.5, only one had NAT with GA (no treatment response), Gem-only as adjuvant for 4 doses. got Gem-NP, patient recurred in 3 months (first retagging scan) MUC5Ac of 3
one with 1.75, PD in 1 year
   One with 1.9, pPD in 9 months
   Rest had Gem based, one had Gem-Cap. PFS was 9
4/7 died or did not get therapy. 3 who had second line had Gem-NP, all of then >1.5
   Post surgery, 12 W (N=32)
Mean MUC5AC 2.9, 0.918 (0.05 to 44.43) (0.559 to 2.45)
CA 19-9, 221, 19 (0-440)
<1 vs. >1—not a great one 15 vs. 17
   PFS 2114 vs. 14 21
O.559, 8 vs. 24
   PFS 15 vs. 21
2.45, 24 vs 8
   PFS. 18 vs. 24

### Example 6: MUC5AC Tissue Analysis

Baseline characteristics. There were 112 matched tumor and serum samples (FIG. 17) from patients treated at the Ohio State University for pancreatic ductal adenocarcinoma (PDA). Ten had metastatic disease at diagnosis or at surgery (1 had microscopic liver metastasis, and 9 were samples from metastatic sites such as lung, liver, omentum, ovary, and bladder). One patient has a neuroendocrine pancreatic tumor, and the other patient's slides provided to us did not have tumor tissue even though a residual tumor was reported in the resected sample.

After removing 12 patients, 100 patients remained in the study who had curative resection for PDA. The median age of diagnosis was 65 years, with 50% males. A documented history of pancreatic cyst or intraductal papillary mucinous neoplasms (IPMN) was in 20%, and all had diabetes mellitus (DM). Neoadjuvant therapy (NAT) was given to 43 patients (36 had FOLFIRINOX, 5 had gemcitabine—nabpaclitaxel (Gem-NP), and 2 had FOLFOX), and the other 57 had upfront surgery (UpS). Neoadjuvant chemoradiation (CRT) was given to 16 patients.

Among UpS patients, 55/57 got systemic adjuvant therapy (3 FOLFIRINOX, 7 Gem-NP, 33 Gem-only, 11Gem/capecitabine (Gem/Cap), and 1 5FU only). The other two patients had adjuvant CRT only (one with Gem and the other with 5FU). Other pathological features noted in the resected samples are detailed in Table 8. T-stage of the patients (70/100 were VIIth TNM staging edition) were reclassified according to the current VIIIth TNM staging edition. There were two T4 patients; one was classified as T2 and the other as T3 to do a size-related analysis.

The pathological response noted on the resected sample in NAT-group reported from the scale of 1-3 based on the treatment response. For practical purposes, patients with extensive tumors with no evident tumor regression or 3 were designated as the no-response (NR) group. Patients with residual tumors with evidence of regression or 2 and near complete response or single cells or small group of cancer cells or 1 were considered as partial-response (PR) or near complete-response (nCR), respectively. The objective response (OR) group refers to patients with PR and nCR. EC-45M1 detection and apical 45M1-H-score were combined and it was referred to as EC-45M1-H-score, which is a composite score.

### MUC5AC Detection in Resected PDA Patients

MUC5AC detection/distribution among the samples is discussed in Table 9 below. Both glycoforms were detected in 96% of the tested samples, and there was 100% overlap for them (i.e., all the samples positive for CLH2 were also positive for 45M1). Four samples were negative for both. Extracellular (EC) 45M1 was detected in 72%, and all had 45M1 and CLH2 expression. CLH2 was detected only in the cytoplasm and NOT in the apical or extracellular (EC) regions. The mature glycoform, 45M1, was detected only in the apical and EC regions. Mean expression levels (H-score) for 45M1 and CLH2 were 148.5 and 145.27, respectively. A positive correlation (≤0.001) exists between the H-scores of these two glycoforms. EC-45M1 was not detected in a quarter (24/96, 14/43 in NAT and 10/57 in Ups groups) of PDAs that expressed 45M1. Intracellular (InC including cytoplasm or apical) MUC5AC is significantly (p<0.001) higher in EC-positive tumors (45M1, 59 vs. 183 and CLH2, 59 vs. 177). The breakdown of MUC5AC expression levels in NAT and UpS groups is detailed in Table 9 and FIG. 15.

The InC MUC5AC expression and EC-MUC5AC detection rates were lower in NAT than UpS group. The mean CLH2 H-score and EC-45M1-detection rates were significantly lower in NAT-group than UpS. The 45M1 H-score was lower with a trend towards significance in NAT-group (Table 9). The tend of significantly higher InC MUC5AC expression in EC-positive tumors was maintained in NAT and UpS groups. The breakdown of the NAT-group was based on the chemotherapy received (Table 19).

### Conclusions

At baseline (diagnosis or treatment naïve, UpS group in our analysis)
   MUC5AC glycoforms are detected in PDAs.
   The EC-45M1-detection is seen in >80% of PDAs.
The 100% concordance between 45M1 and CLH2 detected MUC5AC confirms that immature MUC5AC (CLH2-detected), matures by glycosylation, moves to the apical region, and is released into extracellular space.
EC-MUC5AC detection could be an indication of InC MUC5AC.
Therapy effects MUC5AC expression levels, even EC-45M1 detection irrespective of the response noted in resected sample (assuming that patient did not progress).
   The data shows FOLFIRINOX has more effect than Gem-NP in MUC5AC

### MUC5AC Expression and Pathological Treatment Response.

The population was divided into three groups, OR, NR, and UpS (Table 8), to compare the MUC5AC expression in three populations (Table 10 & 11). It must be noted that despite no appreciable treatment response (NR) in 16/43 patients in NAT-group, they did not have distant metastasis while on NAT or had microscopic metastatic disease in the liver or peritoneum.

The mean CLH2 expression was significantly different (p=0.01), while 45M1 had a strong trend towards significance (p=0.06) among the three groups (FIG. 16). OR and UpS had the lowest and highest expression levels, respectively, for both MUC5AC glycoforms (Table 10). The NR group had levels close to the UpS group and higher than the OR group. On head-to-head comparisons, MUC5AC expression (mean 45M1 and CLH2) was significantly lower in OR than UpS groups, while NR vs. OR and NR vs. UpS were not significantly different.

The proportion of EC-45M detection (positive vs. negative) differs among the three groups (Table 11). The detection rates were significantly different among the three groups, with the OR group having the lowest and the UpS-group with the highest rates. EC-45M1-detection was significantly different on head-to-head comparisons that follow the same trend as 45M1 (Table 10). The difference between OR and UpS was significant, while the other two were not. The 45M1 expression in EC-positive tumors in the three groups was not different. The difference between the OR and NR groups had a trend toward significance (Table 10). The breakdown according to the chemotherapy regimen received between OR vs. NR MUC5AC expression is discussed in Table 20. The results are confounded by using three different chemo regimens (Table 20). A similar trend is noted in FOLFIRNOX-treated patients (FOLFIRNOX vs. UpS, Table 21).

Two FOLFOX patients were EC-45M1 positive and had NR. The Gem-NP group patients with NR (2/5) had lower InC than the PR-group. The numbers are too low to make any conclusions, but the observations were opposite to those noted in the FOLFIRINOX population. For the entire NAT-group, it should be noted that even though perinuclear (CLH2) or apical (45M1) MUC5AC detection in the NR-group is no different than UpS, EC-45M1 detection rates are numerically closer to the OR-group.

About 30% (4/14) of 45M1-positive patients are not producing EC-45M1. In two patients, 45M1 was not expressed (hence, EC-45M1-negative). Resistance mechanisms (to chemotherapy) could include a drop in EC-MUC5AC production while continuing InC production. A drop in EC-45M1 could have helped in the prevention of metastasis, even in the NR group.

### Conclusion

The tissue CLH2 expression correlated with pathological treatment response (OR<NR<UpS) noted in the resected sample.
A larger study could show significant difference in 45M1, but the trend seen in the study is encouraging (p=0.06).
EC-45M1 detection gives an insight into the treatment response if paired with InC MUC5AC level.
   If both InC and Ec drop, there is PR or nCR
With UpS as a control group, preliminary evidence can extrapolate that InC and EC MUC5AC significantly reduce (by ≈36%) if PDA responds to FOLFIRINOX.
   If they do not have a considerable response (NR) but do not progress, InC MUC5AC levels will NOT drop, but EC-MUC5AC production could stop.
   If they progress on FOLFIRINOX, InC-MUC5AC will NOT drop and EC MUC5AC can still be detected.

### MUC5AC Impact of Pathological Features

The impact of MUC5AC was examined by logistic regression analysis (Table 12) in all the patients in the study, irrespective of NAT (N=100). Treatment response (OR vs. NR vs. UpS) was impacted by 45M1 and CLH2 levels and EC-45M1-detection. EC-45M1-detection influenced Premalignant (IPMN and PanIN) lesion identification, while 45M1 and CLH2 levels had a trend toward significance. All 9 patients with IPMN were positive for CLH2, 45M1, and EC-45M1. In PanIN associated tumors (N=55), 54 had 45M1/CLH2 expression and 44/54 had EC-45M1 detection. NAT (yes vs. no) impacted CLH2 levels and EC-451-detection, and there was a tendency towards significance with 45M1 levels. The effect of MUC5AC on the tumor size was analyzed (Table 13). Larger tumors (>2 cm or T2-T3) had significantly higher 45M1 and CLH2 expression. For the UpS group, T1 was very low (N=9) than T2 (N=36) and T3 (N=12). For NAT-group, T1 was 14 with 19 T2 and 10 T3. Numbers were too low to do reasonable analysis, but the trend is the same (T1<T2-T3). A similar trend was observed in the FOLFIRNOX vs. UpS.

To study the impact of baseline MUC5AC expression on pathological features separately, an univariate regression was performed in UpS group (N=57). The impact of EC-45M1-detection on adjacent organ invasion was the only significant (p=0.04) factor. 45M1 and CLH2 did not impact pathological features. In the NAT group (N=43), 45M1 expression levels were significantly related to perineural invasion (PNI) and residual disease (R1 vs. R1-2), while EC-45M1 detection was related to the occurrence of the premalignant lesions (Table 14).

Treatment response vs. tumor size: 88% and 56% (p=0.03) of NR-group and OR-group in NAT-group had tumors >2 cms (T3-T4). The same trend is noted in the FOLFIRINOX group (92% vs. 54%, p=0.02).

### Conclusion

If UpS is considered as the baseline,
   Neoadjuvant treatment can impact MUC5AC expression.
   Larger tumors tend to have higher MUC5AC expression and are mostly to have NR when treated with FOLFIRINOX

### The Impact of MUC5AC on PFS and OS in PDA

In the study population (NAT+UpS), MUC5AC did not impact OS or PFS on univariate (UVA) and multivariate (MVA) analysis (Tables 23, 24, and 25). Treatment response (OR vs. NR vs. UpS) was one of the factors with significance that impacted both PFS and OS by UVA and MVA.

None of the MUC5AC-related factors in the NAT-group impacted PFS or OS on UVA (Table 15). Pathological factors that had an impact were LVI, margin status, and residual disease status. On MVA, 45M1 and CLH2 had an impact (p<0.05) on both PFS and OS, but the hazard ratios (HR) were not impressive. On the other hand, EC-45M1-detection had a trend towards significance for PFS (p=0.07) but HR was impressive (negative vs. positive, HR=0.03). Other factors are discussed in Tables 16, 17, and 18 below.

### Conclusion

Post-neoadjuvant chemotherapy, EC-MUC5AC may impact the recurrence. EC-MUC5AC-negative tumors have >50% risk of recurrence than positive tumors.

### The Expression of MUC5AC in Primary and Distant Metastatic Sites

There were 10 patients with metastatic disease at the time of resection. One patient who had micrometastasis but had primary resection was excluded from this comparison (Table 28). Four of these 9 patients (treatment naïve) received no systemic therapy before the biopsy. The others (N=5) got systemic therapy (2-FOLFIRNIX, 1-FOLFOX, 1-Gem-NP, 1-Gem only). All 9 biopsies were positive for 45M1, EC-45M1, and CLH2. The mean 45M1, EC-45M1(45M1 H-score in EC-positive tumors, value is 0 for EC-negative tumors), and CLH2 expression were 199, 199, and 196, respectively. Among the metastatic sites, the lung has the highest H-scores, and the peritoneum has the least (lung (300)>Other organs (260)>liver (170)>Primary>peritoneum (100)). The 45M1 and CLH2 H scores were similar.

The numbers were too low to study the effect of the treatment on MUC5AC expression in these two groups. Interestingly, when the MUC5AC expression was compared between metastatic sites (N=9) and resected specimens (N=100), CLH2 and EC-45M1 H-scores were significantly higher in metastatic sites than primary tumors (Table 18). Interestingly, the percentage of positive cells with 45M1 and CLH2 were significantly higher in metastatic sites.

### Conclusions

The MUC5AC expression is higher intracellularly than the primary tumor and is always associated with EC-45M1 expression.
MUC5AC is detected (positive) in most PDAs
PDAs that respond to chemotherapy, have lower levels of MUC5AC (CLH2, EC-45M1-detection, and 45M1)
   MUC5AC levels are not different from the baseline if you do not respond.
EC-45M1-detection
   is associated with premalignant lesions (PanIN and IPMN)
Tend to have poor PFS.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
List of methylation markers for diagnosis

| | | Intraductal Papillary |
|---|---|---|
| Epigenetic Markers | Our proposed panel derived from | Mucinous Neoplasm - |
| (DNA-methylation) | the literature for diagnosis. | specific markers. |
| | Diagnostic panel with evidence (n = 68) | BNIP3 CADM1 |
| | ADAMTS1 APC BMP3 BNC1 BRCA1 CCDC181 CCND2 CDKN1C | CLDN5 DISP3 EBF3 |
| | CDKN2B DAPK1 EPB41L3 ESR1 ALKAL1 GATA4 GATA6 GPC3 | EYA4 FOXE1 NXPH1 |
| | HOXA1 HOXA7 IGF1 LRFN5 MAPT MARCHF11 MEIS2 MEST | RELN SLIT2 SOX17 |
| | MGMT MIR-129-2/129 MLH1 MUC2 MYOD1 NPTX2 ONECUT1 | (n = 11) |
| | CDKN2A PANTR1 PCDH10 PENK PGK1 PGR PLAU PRKCB PROX1 | |
| | PXDN RARB RASSF1 RB1 RUNX3 SEPTIN9 SFRP1 SFRP2 SIX3 | |
| | SLC9A3 SOCS1 SPARC SPSB4 CD2 SST SYK TBR1 TEAD1 THBS1 | |
| | TYMS TRIM73 UCHL1 VHL VWC2 YAP1 ZNF781 SEMA5A TFPI2 | |
| | MIR-1292/3P MIR-1292/5P MUC5AC | |
| | Exploratory panel (n = 84) | |
| | ABCB1 ABCB11 ABCB4 ABCB5 ABCC1 ABCC10 ABCC11 ABCC2 | |
| | ABCC3 ABCC5 ABCG2 ACIN1 ALX4 ARID1A ASXL2 BCL2L1 CDA | |
| | CDKN2A CES2 CHFR CTNNB1 CXCL8 DCK DDX23, DKK3 DPYD | |
| | EGFR FOXM1 GSTM1 GSTM2 GSTP1 HIC1 HMGA1 HNF1A HSPA5 | |
| | IGFBP3 ISG15 JAG1 LCN2 LONRF2 MAP2 MAP3K7 MCL1 METTL3 | |
| | MSX2 MUC4 MUC5AC MUTYH MYEOV NEUROG1 NOTCH2 NRP1 | |
| | NT5C1A ONECUT2 POU1F1 PROKR2 PTGES2 PYCARD RRM1 | |
| | RRM2 S100A4 SFN SLC22A2 SLC22A3 SLC22A7 SLC28A1 SLC28A2 | |
| | SLC28A3 SLC29A1 SLC29A3 SLFN11 SMARCA2 SNRPF SOX8 | |
| | TET1 TFAP2E TM4SF1 TNFRSF10C TNFSF10 TSPAN1 VASH2 | |
| | WNT5A ZEB1 | |

TABLE 2
Relationship between MUC5AC, CA19-9, and treatment response.

High vs low

| | nCR | PR | NR | PD |
|---|---|---|---|---|
| MUC5AC | Low | High | Low-stable | High |
| CA 19-9 | Low | Low | Stable -high | High |

nCR—near clear response,
PR—partial response,
NR—no response,
PD—disease progression

TABLE 3
Comparing low and high MUC5AC groups (N = 23) in patients post-NAT*

| | | | |
|---|---|---|---|
| Threshold MUC5AC | ≤0.7 (12) vs. >0.7 (11) | <1 (13) vs. >1 (10) | <1.82 (16) vs. >1.82 (7) |
| in ng/mL (N) | | | |
| nCR vs. PR vs. NR (%) | 17/25/58 vs. 0/55/45 | 15/23/62 vs. 0/60/40 | 12.5/25/63 vs. 0/71/29 (0.09) |
| OR (%) | 42 vs. 55 | 38 vs. 60 | 38 vs. 71 |
| CA 19-9 mean (ng/mL) | 1100 vs. 237 | 1020 vs. 255 | 831 vs. 361 |
| 45M1 H-score/% | 115/38 vs. 161/57 | 127/42 vs. 150/50 | 118/39 vs. 180/60 |
| distribution | | | |
| CLH2 score/% | 115/38 vs. 152/54 | 127/42 vs. 140/50 | 119/41 vs. 163/57 |
| distribution | | | |
| EC-45M1-detection % | 50 vs. 73 | 45 vs 75 | 50 vs. 86 |
| EC-45M1-H-score | 93 vs. 137 | 86 vs. 147 | 85 vs. 176 (0.08) |
| T1 vs T2-T3, T2-3% | 92 vs. 55 (0.04) | 92% vs. 50% (0.02) | 88 vs. 43 (0.02) |
| R0 vs R12, R0% | 33 vs. 82 (0.01) | 62% vs. 20% (0.04) | 30 vs. 100 (0.005) |
| Margin -positive % | 33 vs. 73 | 39 vs. 70 (0.07) | 38 vs. 86 (0.03) |
| Node positive % | 75 vs. 75 | 77% vs 70% | 82 vs. 58 |
| Perineural invasion- | 100 vs. 73 (0.05) | 0 vs. 70 (0.01) | 94 vs. 71 |
| positive % | | | |
| Lymph vascular | 67 vs. 45 | 63 vs. 25 (0.09) | 63 vs. 43 |
| invasion- positive % | | | |
| Progression free | 8.5 vs. 5.5 | 9 vs. 4.5 | 9 vs. 3 (0.04) |
| survival in months | | | |

*if p-value is ≥0.1, it was not reported here.

TABLE 4
Comparing low and high MUC5AC groups (N = 19) in patients treated with FOLFIRINOX*

| | | | |
|---|---|---|---|
| Threshold MUC5AC (N) | ≤1 (11) vs. >1 (8) | ≤0.7 (10) vs. >0.7 (9) | ≤1.82 (14) vs. >1.82 (5) |
| CA 19.9 mean (ng/mL) | 1036 vs. 227 | 1134 vs. 208 | 819 vs. 358 |
| 45M1 H-score | 146 vs. 132 | 134 vs. 147 | 132 vs. 162 |
| CLH2 score | 146 vs. 127 | 134 vs. 142 | 133 vs. 150 |
| EC-45M1-detection % | 45 vs 75 | 50 vs. 67 | 50 vs. 80 |
| EC-45M1-H-score | 101 vs. 127 | 120 vs. 113 | 96 vs. 156 |
| CR vs. PR vs. NR (%) | 18/18/64 vs. 0/63/37 | 10/20/60 vs. 0/55/45 | 14/21/65 vs. 0/80/20 (0.06) |
| OR (%) | 37 vs. 63 | 40 vs. 55 | 36 vs. 80 (0.08) |
| T1 vs T2-T3 , T1-3% | 91% vs. 50% (0.04) | 90 vs. 56 (0.08) | 86 vs. 40 (0.04) |
| R0 vs R12, R1-2% | 37% vs. 75% (0.09) | 30 vs. 78 (0.03) | 36 vs. 100 (0.01) |
| Margin -positive % | 36 vs. 63 | 30 vs. 67 | 36 vs. 80 |
| Node positive % | 73% vs 63% | 70 vs. 67 | 79 vs. 40 |
| Perineural invasion- | 100 vs. 63 (0.02) | 100 vs. 67 (0.04) | 93 vs. 60 |
| positive % | | | |
| Lymph vascular | 63 vs 25 (0.09) | 60 vs. 33 | 57 vs. 20 |
| invasion %, | | | |
| Progression free | 9 vs. 45 | 8 vs. 5 | 9 vs. 3 |
| survival in months | | | |

*if p-value is ≥0.1, it was not reported here.

TABLE 5
Serum MUC5AC predicting treatment response or recurrence.

| | | | Last systemic | |
|---|---|---|---|---|
| | | | therapy dose | |
| | MUC5AC | CA 19-9 | to progression | |
| | (ng/mL) | (ng/mL) | (in weeks) | Comments |
| Surveillance 1 | 3 | 10347 | 57 | PFS was 21 m |
| Surveillance 2 | 1.7 | 3292 | 27 | PFS was 14 m |
| PoAT 1 (Gem) | 2 | Undetectable* | 4 | Just after the |
| | | | | initiation of AT |
| PoAT 2 (Gem) | 1.8 | Undetectable* | 8 | PD just after |
| PoAT 3(Gem) | 0.5 | Undetectable* | 8 | completing 6 m AT |
| PoAT 4 | 0.82 | 54^{#} | 2 | |
| (gem/cap) | | | | |
| PoAT 5 (Gem) | 0.7 | 38^{#} | 4 | |
| PoSL 1 (on | 0.6 | 455\hat{ } | On therapy | TTP on FOLFOX was 9 m, |
| FOLFOX) | | | | 25 weeks after this |
| | | | | MUC5AC level. The |
| | | | | patient was on a clinical |
| | | | | trial with Gem and a |
| | | | | vaccine. |
| PoSL 2 | 24 | 109233 | On therapy | This level was at PD. |
| (on Gem) | | | | Disease was stable for over |
| | | | | 8 m on Gem-NP and 6 m on |
| | | | | Gem before this. |
| PoSL 3 | 19 | 59932 | On therapy | This level was 6 weeks |
| (on Gem-NP) | | | | after PD on the first line |
| | | | | and 2 weeks before PD on |
| | | | | the second line |

*CA 19-9 was always undetectable.
^{#}CA 19-9 was undetectable until that point.
\hat{ }Despite rising CA 19-9, there was no evidence of progression on the imaging in these patients.
PFS—progression-free survival; m—months; AT—adjuvant therapy; PoAT—progression on AT; PoSL—progression on second-line therapy; TTP—time to progression; PD—disease progression; Gem—gemcitabine; NP—nab-paclitaxel; cap—capecitabine.

TABLE 6
Serum MUC5AC and CA 19-9 levels with respect to surgery and recurrence.

| | | Mean MUC5AC | CA 19-9 | Median time |
|---|---|---|---|---|
| | | (in ng/mL) | (in ng/mL) | (in weeks) |
| After | Before recurrence (39) | 2.9 | 405 | 10 |
| surgery | No recurrence (11) | 1.1 | 10 | 11 |
| Before | Before recurrence (27) | 2.75 | 839 | 9 |
| surgery | No recurrence (4) | 0.9 | 148 | 10.5 |

TABLE 7
Serum MUC5AC in the recurrent group while on
gemcitabine-based therapy adjuvant therapy

| Relation to | | MUC5AC | Median time from |
|---|---|---|---|
| chemotherapy | | (in ng/mL) | chemotherapy (in weeks) |
| After | Gem-only (18) | 5.3* | 6.5 |
| | Gem-based (5, | 3 | 4 |
| | NP-3, Cap-2) | | |
| Before | Gem (6) | 1.1 | 2.5 |
| | Gem-based (6, | 1.4 | 2 |
| | NP-4, cap-2) | | |

*one outlier of 44, without it, it is 1.82.

TABLE 8
Pathological features identified on the resected pancreatic tissues.

| Pathological feature | Distribution in percentage |
|---|---|
| Differentiation (G1 vs. | 11 vs 63 |
| G2 vs. G3) | vs. 27 |
| Adjacent organ invasion | 50 |
| identified | |
| Treatment effect noted in | 27 vs. 16 |
| NAT-group (N = 43). | (nCR - 4; PR - 23) |
| OR vs. NR | |
| Tumor size, ≤2 cms (T1) vs. | 23 vs. 55 |
| 2-4 cms (T2) vs. >4 cms (T3) | vs. 22 |
| T-stage, T1 vs. T2 vs. | 23 vs. 54 vs. |
| T3 vs. T4 | 21 vs. 2 |
| Lymph vascular invasion identified | 64 |
| Perineural invasion identified | 80 |
| Margins positive | 29 |
| Residual disease: R0 vs. | 72 vs. 26 |
| R1 vs. R2 | vs. 2 |
| Node positivity: N0 vs. N1-2 | 20 vs. 71 |
| Premalignant lesion identified | 54 vs. 9 |
| on the resected sample: PanIN | vs. 37 |
| vs. IPMN vs. no lesion identified | |

OR—objective response, NR—nor response, nCR—near complete response, PR—partial response, PanIN—pancreatic intraepithelial neoplasia, IPMN—intraductal papillary mucinous neoplasms.

TABLE 9
Distribution of MUC5AC expression in the tested samples

| | Mature | | Immature |
|---|---|---|---|
| | Detection rate in %/H-score median (range)/H-score mean | | |
| Population (N) | 45M1 | EC-45M1* | CLH2 |
| Total (100) | 96%/150(0-300)/148.5 | 72% | 96%/150(0-300)/145.2 |
| NAT (43) | 91%/120(0-270)/129 | 58% | 91%/100(0-270)/122 |
| UpS (57) | 100%/180 (0-300)/164 | 82% | 100%/180(0-300)/163 |
| NAT vs. UpS (p-value) | 0.06 | 0.007 | 0.02 |

*Detection rates NAT—neoadjuvant therapy group,
UpS—upfront surgery group,
EC—extracellular

TABLE 10
Comparing 45M1 and CLH2 among OR, NR, and UpS groups (N = 100).

| | Mean H-scores, OR vs. NR vs. UpS | Head-to-head comparisons |
|---|---|---|
| 45M1 | 113.3 vs. 154.38 vs. 163.6 (p = 0.0612) | OR vs. UpS (p = 0.0498) |
| | | NR vs. UpS (p = 0.9308) |
| | | OR vs. NR (p = 0.3245) |
| EC-45M1 | 100 vs. 121 vs. 151 (0.1) | OR vs. UpS (p = 0.8028) |
| (EC positive + | | NR vs. UpS (p = 0.5648) |
| H score) | | OR vs. NR (p = 0.0969) |
| CLH2 | 104.07 vs. 151.88 vs. 162.93 (p = 0.0184) | OR vs. UpS (p = 0.0140) |
| | | NR vs. UpS (p = 0.8969) |
| | | OR vs. NR (p = 0.2015) |

OR—objective response,
NR—no response,
UpS—upfront surgery.
EC—extracellular

TABLE 11
Extracellular MUC5AC expression distribution in all the patients (N = 100).

| Groups | % positive | p-value* | Head-to-head comparisons |
|---|---|---|---|
| OR | 56 | OR vs. NR vs. UpS = 0.02 | OR vs. UpS (p = 0.0155) |
| NR | 63 | | NR vs. UpS (p = 0.1008) |
| UpS | 82 | | OR vs. NR (p = 0.7548) |
| Total | 72 | | |

OR—objective response,
NR—no response,
UpS—upfront surgery

TABLE 12
Univariate logistic regression model for MUC5AC
expression in all the patients (N = 100).

| | | | EC-45M1- | EC-Positive + |
|---|---|---|---|---|
| | 45M1 | CLH2 | detection | 45M1 H-score |
| Pathological feature | p-value | p-value | p-value | p-value |
| Path diff G1-2 vs G3 | 0.6866 | 0.8559 | 0.4358 | 0.2984 |
| Adjacent organ invasion | 0.6200 | 0.8741 | 0.1841 | 0.1496 |
| Treatment effect (OR vs. NR vs. | 0.0317 | 0.0112 | 0.0081 | 0.0379 |
| UpS) | | | | |
| Lymph vascular invasion | 0.8954 | 0.9811 | 0.3744 | 0.8347 |
| Perineural invasion | 0.4129 | 0.4802 | 0.8238 | 0.8254 |
| Margins | 0.5935 | 0.8658 | 0.5832 | 0.8341 |
| Residual disease | 0.2977 | 0.6145 | 0.4018 | 0.4172 |
| R0 vs R1-2 | 0.2971 | 0.6141 | 0.3642 | 0.4165 |
| N0 vs N1-N1 | 0.9004 | 0.8697 | 0.5832 | 0.4742 |
| Premalignant (yes vs. no) | 0.0585 | 0.0643 | 0.0007 | 0.0088 |
| Neoadjuvant CRT | 0.3952 | 0.2876 | 0.3592 | 0.4597 |
| Neoadjuvant therapy (yes vs. no) | 0.0612 | 0.0264 | 0.0088 | 0.0441 |
| Site of recurrence | 0.1381 | 0.1405 | 0.6321 | 0.2686 |

NR—No response,
OR—objective response,
UpS—upfront surgery

TABLE 13
Univariate logistic regression model for MUC5AC expression
in all the patients (N = 100) for tumor size.

| MUC5AC glycoform | T1 vs. T2 vs. T3 | T1 vs T2-3 |
|---|---|---|
| 45M1, H-score & % | 115 vs. 161 vs. 153 (0.1) & | 115 vs. 159 (0.03) & |
| distribution | 39 vs. 54 vs. 52 (0.1) | 39 vs. 53 (0.04) |
| CLH2, H-score & % | 112 vs. 154 vs. 157 (0.1) & | 112 vs. 155 (0.03) & |
| distribution | 41 vs. 52 vs, 53 (0.2) | 41 vs. 52 (0.08) |
| EC, H-score & EC-percentage | 89 vs. 151 vs. 130 (0.05) & | 89 vs. 145 (0.01) & |
| positivity | 61 vs. 78 vs. 68 (0.2) | 61 vs. 75 (0.1) |

EC—extracellular

TABLE 14
Univariate logistic regression model for MUC5AC
expression in neoadjuvant group (N = 43).

| | | | | EC-45M1-H- |
|---|---|---|---|---|
| | 45M1 | CLH2 | Extracellular | score |
| Pathological feature | p-value | p-value | p-value | p-value |
| Path diff G1-2 vs G3 | 0.8699 | 0.9096 | 0.6690 | 0.4631 |
| Adjacent organ invasion | 0.3616 | 0.1590 | 0.8970 | 0.7297 |
| Treatment effect No response vs. CR | 0.1842 | 0.1129 | 0.6558 | 0.5419 |
| or PR vs No therapy | | | | |
| Lymphovascular invasion | 0.6087 | 0.7198 | 0.9769 | 0.8538 |
| Perineural invasion | 0.0419 | 0.0662 | 0.5529 | 0.1203 |
| Margins | 0.0707 | 0.1182 | 0.1855 | 0.2252 |
| R0 vs R1-2 | 0.0300 | 0.0811 | 0.0710 | 0.0658 |
| T-stage (70 - VIIth and 30 - VIIIth | 0.0921 | 0.0650 | 0.1804 | 0.2056 |
| edition) | | | | |
| N0 vs N1-N1 | 0.7511 | 0.6899 | 0.4089 | 0.4653 |
| Premalignant yes or no | 0.7749 | 0.9116 | 0.0136 | 0.2844 |
| Neoadjuvant CRT | 0.9160 | 0.9457 | 0.6558 | 0.7519 |
| Site of recurrence | 0.3041 | 0.3106 | 0.7705 | 0.5442 |

TABLE 15
Univariate analysis of clinicopathological features
for survival in neoadjuvant group (N = 43).

| | Time to | Overall |
|---|---|---|
| | Recurrence | Survival p- |
| Clinicopathological feature | p-value | value |
| Path diff G1-2 vs G3 | 0.1054 | 0.1165 |
| ADJACENT ORGAN INVASION | 0.1309 | 0.4349 |
| Treatment effect No response vs. CR or | 0.8751 | 0.9958 |
| PR vs No therapy | | |
| Lymphovascular invasion | 0.0020 | 0.0359 |
| Perineural invasion | 0.1629 | 0.2652 |
| Margins | 0.0007 | <.0001 |
| R0 vs R1-2 | 0.0135 | 0.0294 |
| T-stage(70 - VIIth and 30 - VIIIth | 0.2764 | 0.0301 |
| edition) | | |
| N0 vs N1-N1 | 0.5599 | 0.8036 |
| Premalignant yes or no | 0.7864 | 0.2314 |
| 45M1 | 0.6711 | 0.8130 |
| Positive + 45M1 | 0.9251 | 0.7686 |
| CLH2 | 0.6847 | 0.8217 |
| Extracellular MUC5AC, positive vs. | 0.5703 | 0.9961 |
| negative | | |
| Neoadjuvant CRT | 0.8484 | 0.9286 |
| Site of recurrence | 0.0656 | 0.7305 |

TABLE 16
Multivariate analysis of the clinicopathological features influencing
progression-free survival in neoadjuvant therapy group (N = 47).
Analysis of Maximum Likelihood Estimates

| | | | Parameter | Standard | Chi- | | Hazard | |
|---|---|---|---|---|---|---|---|---|
| Parameter | | DF | Estimate | Error | Square | Pr > ChiSq | Ratio | Label |
| _5M1_{——}H_score | | 1 | −0.04256 | 0.01828 | 5.4187 | 0.0199 | 0.958 | 45M1 -H- |
| | | | | | | | | score |
| CLH2_{——}H_score | | 1 | 0.03887 | 0.01861 | 4.3625 | 0.0367 | 1.040 | CLH2 -H- |
| | | | | | | | | score |
| Extracellular_{—} | Negative | 1 | −1.17781 | 0.67120 | 3.0792 | 0.0793 | 0.308 | Extracellular |
| positi | | | | | | | | positive vs |
| | | | | | | | | negative |
| | | | | | | | | Negative |
| Path_diff_G1_{—} | G1-2 | 1 | −1.45042 | 0.55214 | 6.9008 | 0.0086 | 0.234 | Path diff |
| 2_vs_G3 | | | | | | | | G1-2 vs G3 |
| | | | | | | | | G1-2 |
| ADJACENT_{—} | NO | 1 | −0.11570 | 0.56687 | 0.0417 | 0.8383 | 0.891 | ADJACENT |
| ORGAN_INVAS | | | | | | | | ORGAN |
| | | | | | | | | INVASION |
| | | | | | | | | NO |
| Treatment_{—} | CR or | 1 | 0.08042 | 0.67312 | 0.0143 | 0.9049 | 1.084 | Treatment |
| effect_No_{—} | PR | | | | | | | effect No |
| | | | | | | | | response vs |
| | | | | | | | | CR or PR vs |
| | | | | | | | | No therapy |
| | | | | | | | | CR or PR |
| LYMPHO | NO | 1 | −1.36342 | 0.61470 | 4.9197 | 0.0266 | 0.256 | LYMPHOVASCULAR |
| VASCULAR_{—} | | | | | | | | INVASION |
| INVAS | | | | | | | | (LVI) NO |
| PERI_{—} | NO | 1 | −0.40143 | 0.86112 | 0.2173 | 0.6411 | 0.669 | PERI |
| NEURAL_{—} | | | | | | | | NEURAL |
| INVASION | | | | | | | | INVASION |
| | | | | | | | | NC |
| Margins | Negative | 1 | −2.19413 | 0.72615 | 9.1300 | 0.0025 | 0.111 | Margins |
| | | | | | | | | Negative |
| R0_vs_R1_2 | R0 | 1 | −0.04143 | 0.78528 | 0.0028 | 0.9579 | 0.959 | R0 vs R1-2 |
| | | | | | | | | R0 |
| T_stage | T1 | 1 | −1.66400 | 1.37080 | 1.4735 | 0.2248 | 0.189 | T-stage T1 |
| T_stage | T2 | 1 | −3.35535 | 1.20057 | 7.8108 | 0.0052 | 0.035 | T-stage T2 |
| T_stage | T3 | 1 | −3.16954 | 1.16143 | 7.4475 | 0.0064 | 0.042 | T-stage T3 |
| N0_vs_N1_N1 | N0 | 1 | −0.50873 | 0.63131 | 0.6494 | 0.4203 | 0.601 | N0 vs N1- |
| | | | | | | | | N1 N0 |
| Premalignant_{—} | None | 1 | −0.51815 | 0.60470 | 0.7342 | 0.3915 | 0.596 | Premalignant |
| yes_or_{—} | | | | | | | | yes or no |
| | | | | | | | | None |
| NAT_CRT_{—} | Had | 1 | −1.56103 | 0.51712 | 9.1127 | 0.0025 | 0.210 | NAT CRT |
| Yes_vs_No | NAT | | | | | | | Yes vs No |
| | CRT | | | | | | | Had NAT |
| | | | | | | | | CRT |
| Site_of_{—} | Distant | 1 | 2.72011 | 0.83320 | 10.6581 | 0.0011 | 15.182 | Site of |
| recurrence_f | met | | | | | | | recurrence |
| | | | | | | | | for anaysis |
| | | | | | | | | Distant met |
| Site_of_{—} | Local | 1 | 3.39060 | 1.01227 | 11.2192 | 0.0008 | 29.684 | Site of |
| recurrence_f | | | | | | | | recurrence |
| | | | | | | | | for anaysis |
| | | | | | | | | Local |

TABLE 17
Multivariate analysis of the clinicopathological features influencing Overall Survival.
Analysis of Maximum Likelihood Estimates

| | | | Parameter | Standard | Chi- | | Hazard | |
|---|---|---|---|---|---|---|---|---|
| Parameter | | DF | Estimate | Error | Square | Pr > ChiSq | Ratio | Label |
| _5M1_{——}H_{—} | | 1 | −0.04115 | 0.01897 | 4.7052 | 0.0301 | 0.960 | 45M1 - |
| score | | | | | | | | H-score |
| CLH2_{——}H | | 1 | 0.04174 | 0.01939 | 4.6361 | 0.0313 | 1.043 | CLH2 - |
| score | | | | | | | | H-score |
| Extracellular_{—} | Negative | 1 | −0.96765 | 0.76884 | 1.5841 | 0.2082 | 0.380 | Extracellular |
| positi | | | | | | | | positive |
| | | | | | | | | vs |
| | | | | | | | | negative |
| | | | | | | | | Negative |
| Path_diff_{—} | G1-2 | 1 | −1.46747 | 0.57938 | 6.4151 | 0.0113 | 0.231 | Path diff |
| G1_2_{—} | | | | | | | | G1-2 VS |
| vs_G3 | | | | | | | | G3 G1-2 |
| ADJACENT_{—} | NO | 1 | 0.52048 | 0.52125 | 0.9970 | 0.3180 | 1.683 | ADJACENT |
| ORGAN_{—} | | | | | | | | ORGAN |
| INVAS | | | | | | | | INVASION |
| | | | | | | | | NO |
| Treatment_{—} | CR or | 1 | 0.58533 | 0.64621 | 0.8205 | 0.3650 | 1.796 | Treatment |
| effect_No_{—} | PR | | | | | | | effect |
| | | | | | | | | No |
| | | | | | | | | response |
| | | | | | | | | vs CR or |
| | | | | | | | | PR vs No |
| | | | | | | | | therapy |
| | | | | | | | | CR or PR |
| LYMPHO | NO | 1 | −0.88442 | 0.63097 | 1.9647 | 0.1610 | 0.413 | LYMPHOVASCULAR |
| VASCULAR_{—} | | | | | | | | INVASION |
| INVAS | | | | | | | | (LVI) |
| | | | | | | | | NO |
| PERI_{—} | NO | 1 | 0.00559 | 0.88344 | 0.0000 | 0.9950 | 1.006 | PERI |
| NEURAL_{—} | | | | | | | | NEURAL |
| INVASION | | | | | | | | INVASION |
| | | | | | | | | NO |
| Margins | Negative | 1 | −3.12623 | 0.84212 | 13.7814 | 0.0002 | 0.044 | Margins |
| | | | | | | | | Negative |
| R0_vs_{—} | R0 | 1 | 1.29959 | 0.79666 | 2.6611 | 0.1028 | 3.668 | R0 vs R1- |
| R1_2 | | | | | | | | 2 R0 |
| T_stage | T1 | 1 | −2.82990 | 1.82527 | 2.4037 | 0.1210 | 0.059 | T-stage |
| | | | | | | | | T1 |
| T_stage | T2 | 1 | −3.41823 | 1.33543 | 6.5518 | 0.0105 | 0.033 | T-stage |
| | | | | | | | | T2 |
| T_stage | T3 | 1 | −3.17918 | 1.30869 | 5.9014 | 0.0151 | 0.042 | T-stage |
| | | | | | | | | T3 |
| N0_vs_N1 | N0 | 1 | −0.56572 | 0.69635 | 0.6600 | 0.4166 | 0.568 | N0 vs |
| N1 | | | | | | | | N1-N1 |
| | | | | | | | | N0 |
| Premalignant_{—} | None | 1 | 0.35303 | 0.56874 | 0.3853 | 0.5348 | 1.423 | Premalignant |
| yes_or_{—} | | | | | | | | yes |
| | | | | | | | | or no |
| | | | | | | | | None |
| NAT_CRT_{—} | Had | 1 | −1.15069 | 0.50141 | 5.2666 | 0.0217 | 0.316 | NAT |
| Yes_{—} | NAT | | | | | | | CRT Yes |
| vs_No | CRT | | | | | | | vs No |
| | | | | | | | | Had NAT |
| | | | | | | | | CRT |
| Site_of_{—} | Distant | 1 | 0.99846 | 0.90188 | 1.2257 | 0.2683 | 2.714 | Site ofe |
| recurrence_{—} | met | | | | | | | recurrenc |
| f | | | | | | | | for |
| | | | | | | | | anaysis |
| | | | | | | | | Distant |
| | | | | | | | | met |
| Site_of_{—} | Local | 1 | 1.92698 | 1.11197 | 3.0031 | 0.0831 | 6.869 | Site of |
| recurrence_{—} | | | | | | | | recurrence |
| f | | | | | | | | for |
| | | | | | | | | anaysis |
| | | | | | | | | Local |

In UpS group, 45M1, CLH2, and EC-45M1-detection did not impact PFS or OS in UVA and MVA. Pathological factors that impacted LVI, margin-status, nide-positivity, site of recurrence and margin status impacted them on UVA. On MVA, PNI and margin status affected PFS, and PNI, associated premalignant lesion, and site of recurrence impacted the OS.

TABLE 18
Comparing MUC5AC expression between metastatic and primary tumors.

| | 45M1 | CLH2 | EC-45M1 |
|---|---|---|---|
| | Metastatic sites (N = 9) vs. resected primary sites (N = 100) (p-value) | | |
| H-score | 199 vs. 149 | 196 vs. 145 | 199 vs. 132 |
| | (0.05) | (0.04) | (0.02) |
| % distribution | 68 vs. 50 (0.03) | 67 vs. 50 (0.03) | NA |
| Positive | 100 vs. 96* | 100 vs. 96* | 100 vs. 72 |
| percentage | | | (0.06) |

EC—extracellular,
NA—not applicable;
*p-value > 0.1

TABLE 19
Breakdown of neoadjuvant therapy group (N = 43) based on treatment.

| | Detection rate in %/H-score median (range)/H-score mean | | |
|---|---|---|---|
| Chemotherapy (N) | 45M1 | EC-45M1* | CLH2 |
| FOLFIRINOX (36) | 88%/90(0-270)/124 | 56% | 88%/90(0-270)//120 |
| FOLFOX (2) | 100%/240(210-270)/240 | 100% | 100%/270/240 |
| Gem-NP (5) | 100%/150 (10-210)/116 | 60% | 100%/120(0- |
| | | | 150)/90 |
| 5FU (38) | 89%/105 (0-270)/130 | 58% | 89%/95 (0-270)/126 |
| Gem (5) | 100%/150 (10-210)/116 | 60% | 100%/120(0- |
| | | | 150)/90 |
| UpS (57) | 100%/180 (0-300)/164 | 82% | 100%/180(0- |
| | | | 300)/163 |

*Detection rates Gem—gemcitabine,
5FU—5flurouracil,
NP—nab-paclitaxel,
Ups—upfront surgery

TABLE 20
Breakdown of neoadjuvant therapy
group (N = 43) based on treatment

| | Objective response vs. no response group^{p} | | |
|---|---|---|---|
| | H-score mean | | |
| | 45M1 | EC-45M1* | CLH2 |
| FOLFIRINOX (36) | 110 vs.153 | 54 vs. 58/96 vs. 12 | 105 vs. 149 |
| FOLFOX (2)^{#} | 240 | 100/240 | 240 |
| Gem-NP (5) | 140 vs. 80 | 67 vs. 50/130 vs. 5 | 97 vs. 80 |
| 5FU-based | 110 vs. 165 | 54 vs. 64/107 vs. | 105 vs. 165 |
| Gem-based | 140 vs. 80 | 137 67 vs. 50/130 | 97 vs. 80 |
| | | vs. 5 | |

^{p}p value of all the comparisons were >0.1.
*Detection rates,
^{#}two patients had NR,
Gem—gemcitabine,
5FU—5flurouracil,
NP—nab-paclitaxel,
Ups—upfront surgery

TABLE 21
Breakdown of neoadjuvant therapy
group (N = 43) based on treatment.

| | Mean H-scores, OR vs. NR vs. UpS (p-value) |
|---|---|
| 45M1 | 110 vs. 153 vs. 164 (0.0514) |
| EC-45M1 | 96 vs. 120 vs. 151 (0.08) |
| EC-45M1* | 54 vs. 58 vs. 82 (0.01) |
| CLH2 | 105 vs. 149 vs. 163 (0.0281) |

*Detection rates,

TABLE 22
Univariate analysis of the clinicopathological
features influencing the outcomes (N = 100).

| | Progression-free | Overall |
|---|---|---|
| | survival | Survival |
| Pathological feature | p-value | p-value |
| Path diff G1-2 vs G3 | 0.1244 | 0.0309 |
| Adjacent organ invasion | 0.0080 | 0.0160 |
| Treatment effect No response vs. CR | 0.0038 | 0.0073 |
| or PR vs No therapy | | |
| LYMPH VASCULAR INVASION | 0.0085 | 0.0126 |
| PERI NEURAL INVASION | 0.0017 | 0.0037 |
| Margins | <.0001 | <.0001 |
| Residual disease | <.0001 | 0.0010 |
| R0 vs R1-2 | <.0001 | 0.0002 |
| T-stage | 0.0106 | 0.0003 |
| N0 vs N1-N1 | 0.0012 | 0.0044 |
| Premalignant yes or no | 0.1414 | 0.2679 |
| 45M1 | 0.6035 | 0.5252 |
| Positive + 45M1 | 0.9409 | 0.8797 |
| CLH2 | 0.6637 | 0.6974 |
| Extracellular MUC5AC, positive vs. | 0.7766 | 0.9525 |
| negative | | |
| Neoadjuvant CRT | 0.0571 | 0.0470 |
| Neoadjuvant therapy | 0.0010 | 0.0020 |
| Site of recurrence | <.0001 | 0.0003 |

TABLE 23
Multivariate analysis of the clinicopathological features influencing the outcomes (N = 100) for PFS.
Analysis of Maximum Likelihood Estimates

| | | | Parameter | Standard | Chi- | | Hazard | |
|---|---|---|---|---|---|---|---|---|
| Parameter | | DF | Estimate | Error | Square | Pr > ChiSq | Ratio | Label |
| _5M1_{——}H_score | | 1 | −0.00652 | 0.00584 | 1.2470 | 0.2641 | 0.993 | 45M1 -H-score |
| CLH2_{——}H_score | | 1 | 0.00610 | 0.00577 | 1.1164 | 0.2907 | 1.006 | CLH2 -H-score |
| Extracellular_positi | Negative | 1 | −0.05766 | 0.40162 | 0.0206 | 0.8858 | 0.944 | Extracellular positive |
| | | | | | | | | vs negative Negative |
| Path_diff_G1_2_{—} | G1-2 | 1 | −0.60238 | 0.28654 | 4.4194 | 0.0355 | 0.548 | Path diff G1-2 vs G3 |
| vs_G3 | | | | | | | | G1-2 |
| ADJACENT_{—} | NO | 1 | −0.05575 | 0.28138 | 0.0393 | 0.8429 | 0.946 | ADJACENT ORGAN |
| ORGAN_INVAS | | | | | | | | INVASION NO |
| Treatment_{—} | CR or PR | 1 | 1.84505 | 0.46394 | 15.8157 | <.0001 | 6.328 | Treatment effect No |
| effect_No_{—} | | | | | | | | response vs CR or PR |
| | | | | | | | | vs No therapy CR or |
| | | | | | | | | PR |
| Treatment_{—} | No response | 1 | 1.95645 | 0.45839 | 18.2166 | <.0001 | 7.074 | Treatment effect No |
| effect_No_{—} | | | | | | | | response vs CR or PR |
| | | | | | | | | vs No therapy No |
| | | | | | | | | response |
| LYMPHO | NO | 1 | −0.98693 | 0.39635 | 6.2003 | 0.0128 | 0.373 | LYMPHOVASCULAR |
| VASCULAR_{—} | | | | | | | | INVASION (LVI) NO |
| INVAS | | | | | | | | |
| PERI_NEURAL_{—} | NO | 1 | −0.64124 | 0.44106 | 2.1137 | 0.1460 | 0.527 | PERI NEURAL |
| INVASION | | | | | | | | INVASION NO |
| Margins | Negative | 1 | −0.79700 | 0.43556 | 3.3482 | 0.0673 | 0.451 | Margins Negative |
| R0_vs_R1_2 | R0 | 1 | 0.12592 | 0.46027 | 0.0748 | 0.7844 | 1.134 | R0 vs R1-2 R0 |
| T_stage | T1 | 1 | −3.64220 | 1.12264 | 10.5257 | 0.0012 | 0.026 | T-stage T1 |
| T_stage | T2 | 1 | −3.20148 | 1.01023 | 10.0429 | 0.0015 | 0.041 | T-stage T2 |
| T_stage | T3 | 1 | −3.44430 | 0.99463 | 11.9916 | 0.0005 | 0.032 | T-stage T3 |
| N0_vs_N1_N1 | N0 | 1 | −0.17624 | 0.37374 | 0.2224 | 0.6372 | 0.838 | N0 vs N1-N1 N0 |
| Premalignant_{—} | None | 1 | −0.56911 | 0.32315 | 3.1016 | 0.0782 | 0.566 | Premalignant yes or no |
| yes_or_{—} | | | | | | | | None |
| NAT_yes_{—} | NONE | 0 | 0 | • | • | • | • | NAT yes vs No NONE |
| vs_No | | | | | | | | |
| NAT_CRT_{—} | Had NAT | 1 | −0.90574 | 0.42188 | 4.6092 | 0.0318 | 0.404 | NAT CRT Yes vs No |
| Yes_vs_No | CRT | | | | | | | Had NAT CRT |
| Site_of_{—} | Distant met | 1 | 2.10177 | 0.46205 | 20.6918 | <.0001 | 8.181 | Site of recurrence for |
| recurrence_f | | | | | | | | anaysis Distant met |
| Site_of_{—} | Local | 1 | 2.25898 | 0.51172 | 19.4873 | <.0001 | 9.573 | Site of recurrence for |
| recurrence_f | | | | | | | | anaysis Local |

TABLE 24
Multivariate analysis of the clinicopathological features influencing the outcomes (N = 100) for overall survival
Analysis of Maximum Likelihood Estimates

| | | | Parameter | Standard | Chi- | Pr > | Hazard | |
|---|---|---|---|---|---|---|---|---|
| Parameter | | DF | Estimate | Error | Square | ChiSq | Ratio | Label |
| _5M1_{——}H_score | | 1 | 0.0005108 | 0.00723 | 0.0050 | 0.9437 | 1.001 | 45M1 -H- |
| | | | | | | | | score |
| CLH2_{——}H_score | | 1 | 0.00102 | 0.00725 | 0.0199 | 0.8878 | 1.001 | CLH2 -H- |
| | | | | | | | | score |
| Extracellular_{—} | Negative | 1 | −0.16300 | 0.42366 | 0.1480 | 0.7004 | 0.850 | Extracellular |
| Positi | | | | | | | | positive |
| | | | | | | | | vs |
| | | | | | | | | negative |
| | | | | | | | | Negative |
| Path_diff_G1_2_{—} | G1-2 | 1 | −0.77635 | 0.30621 | 6.4279 | 0.0112 | 0.460 | Path diff |
| vs_G3 | | | | | | | | G1-2 vs |
| | | | | | | | | G3 G1-2 |
| ADJACENT_{—} | NO | 1 | −0.20982 | 0.28848 | 0.5290 | 0.4670 | 0.811 | ADJACENT |
| ORGAN_INVAS | | | | | | | | ORGAN |
| | | | | | | | | INVASION |
| | | | | | | | | NO |
| Treatment_effect_{—} | CR or | 1 | 1.45959 | 0.43192 | 11.4199 | 0.0007 | 4.304 | Treatment |
| No_{—} | PR | | | | | | | effect No |
| | | | | | | | | response |
| | | | | | | | | vs CR or |
| | | | | | | | | PR vs No |
| | | | | | | | | therapy |
| | | | | | | | | CR or PR |
| Treatment_effect_{—} | No | 1 | 1.15486 | 0.42834 | 7.2689 | 0.0070 | 3.174 | Treatment effect No |
| No_{—} | response | | | | | | | response vs CR or |
| | | | | | | | | PR vs No therapy |
| | | | | | | | | No response |
| LYMPHO | NO | 1 | −0.53294 | 0.37988 | 1.9682 | 0.1606 | 0.587 | LYMPHOVASCULAR |
| VASCULAR_{—} | | | | | | | | INVASION |
| INVAS | | | | | | | | (LVI) NO |
| PERI_NEURAL_{—} | NO | 1 | −0.45601 | 0.43224 | 1.1131 | 0.2914 | 0.634 | PERI NEURAL |
| INVASION | | | | | | | | INVASION NO |
| Margins | Negative | 1 | 1.67144 | 0.43589 | 14.7033 | 0.0001 | 0.188 | Margins Negative |
| R0_vs_R1_2 | R0 | 1 | 1.01948 | 0.44033 | 5.3603 | 0.0206 | 2.772 | R0 vs R1-2 R0 |
| T_stage | T1 | 1 | −4.50226 | 1.26543 | 12.6586 | 0.0004 | 0.011 | T-stage T1 |
| T_stage | T2 | 1 | −3.82260 | 1.11260 | 11.8043 | 0.0006 | 0.022 | T-stage T2 |
| T_stage | T3 | 1 | −4.06577 | 1.09659 | 13.7467 | 0.0002 | 0.017 | T-stage T3 |
| N0_vs_N1_N1 | N0 | 1 | −0.26710 | 0.36559 | 0.5338 | 0.4650 | 0.766 | N0 vs N1-N1 N0 |
| Premalignant_{—} | None | 1 | −0.27979 | 0.29820 | 0.8804 | 0.3481 | 0.756 | Premalignant yes or |
| yes_or_{—} | | | | | | | | no None |
| NAT_yes_{—} | NONE | 0 | 0 | • | • | • | • | NAT yes vs No |
| vs_No | | | | | | | | NONE |
| NAT_CRT_{—} | Had NAT | 1 | −0.74454 | 0.42480 | 3.0718 | 0.0797 | 0.475 | NAT CRT Yes vs No |
| Yes_vs_No | CRT | | | | | | | Had NAT CRT |
| Site_of_{—} | Distant | 1 | 1.31475 | 0.46913 | 7.8542 | 0.0051 | 3.724 | Site of recurrence |
| recurrence_f | met | | | | | | | for anaysis Distant |
| | | | | | | | | met |
| Site_of_{—} | Local | 1 | 1.40777 | 0.51982 | 7.3341 | 0.0068 | 4.087 | Site of recurrence |
| recurrence_f | | | | | | | | for anaysis Local |

TABLE 25
Univariate analysis of the clinicopathological features influencing
the outcomes in upfront surgery group (N = 57).

| | Time to | Overall |
|---|---|---|
| | Recurrence | Survival |
| Pathological feature | p-value | p-value |
| Path diff G1-2 vs G3 | 0.3791 | 0.1300 |
| ADJACENT ORGAN INVASION | 0.0276 | 0.0186 |
| Treatment effect No response vs. | NA | NA |
| CR or PR vs No therapy | | |
| Lymphovascular invasion | 0.0051 | 0.0035 |
| Perineural invasion | 0.0044 | 0.0053 |
| Margins | 0.0040 | 0.0109 |
| Residual disease | 0.0120 | 0.0383 |
| R0 vs R1-2 | 0.0120 | 0.0383 |
| T-stage | 0.1327 | 0.1125 |
| N0 vs N1-N1 | 0.0003 | 0.0014 |
| Premalignant yes or no | 0.7420 | 0.3335 |
| 45M1 | 0.3410 | 0.3844 |
| Positive + 45M1 | 0.4899 | 0.5013 |
| CLH2 | 0.3162 | 0.5012 |
| Extracellular MUC5AC, positive vs. | 0.8573 | 0.5139 |
| negative | | |
| Neoadjuvant CRT | NA | NA |
| Neoadjuvant therapy | NA | NA |
| Site of recurrence | <.0001 | 0.0006 |

TABLE 26
Multivariate analysis of the clinicopathological features influencing
the outcomes in upfront surgery group for PFS (N = 57).
Analysis of Maximum Likelihood Estimates

| | | | Parameter | Standard | Chi- | | Hazard | |
|---|---|---|---|---|---|---|---|---|
| Parameter | | DF | Estimate | Error | Square | Pr > ChiSq | Ratio | Label |
| _5M1_{——}H_score | | 1 | −0.00871 | 0.00936 | 0.8653 | 0.3523 | 0.991 | 45M1 -H-score |
| CLH2_{——}H_score | | 1 | 0.01112 | 0.00979 | 1.2913 | 0.2558 | 1.011 | CLH2 -H-score |
| Extracellular_{—} | Negative | 1 | 0.35029 | 0.60959 | 0.3302 | 0.5655 | 1.419 | Extracellular positive |
| Positi | | | | | | | | vs negative Negative |
| Path_diff_G1_{—} | G1-2 | 1 | −0.17417 | 0.42643 | 0.1668 | 0.6830 | 0.840 | Path diff G1-2 vs G3 |
| 2_vs_G3 | | | | | | | | G1-2 |
| ADJACENT_{—} | NO | 1 | −0.23210 | 0.42915 | 0.2925 | 0.5886 | 0.793 | ADJACENT ORGAN |
| ORGAN_INVAS | | | | | | | | INVASION NO |
| LYMPHO | NO | 1 | −1.14748 | 0.81391 | 1.9876 | 0.1586 | 0.317 | LYMPHOVASCULAR |
| VASCULAR_{—} | | | | | | | | INVASION (LVI) |
| INVAS | | | | | | | | NO |
| PERI_NEURAL_{—} | NO | 1 | −1.64043 | 0.75538 | 4.7161 | 0.0299 | 0.194 | PERI NEURAL |
| INVASION | | | | | | | | INVASION NO |
| Margins | Negative | 1 | −0.93545 | 0.84317 | 1.2309 | 0.2672 | 0.392 | Margins Negative |
| R0_vs_R1_2 | R0 | 1 | 0.94685 | 0.92286 | 1.0527 | 0.3049 | 2.578 | R0 vs R1-2 R0 |
| T_stage | T1 | 1 | −1.46417 | 1.29187 | 1.2845 | 0.2571 | 0.231 | T-stage T1 |
| T_stage | T2 | 1 | 1.25223 | 0.62741 | 3.9835 | 0.0459 | 3.498 | T-stage T2 |
| N0_vs_N1_N1 | N0 | 1 | −0.81864 | 0.76324 | 1.1504 | 0.2835 | 0.441 | N0 vs N1-N1 N0 |
| Premalignant_{—} | None | 1 | −0.71198 | 0.49973 | 2.0299 | 0.1542 | 0.491 | Premalignant yes or no |
| yes_or_{—} | | | | | | | | None |
| Site_of_{—} | Distant | 1 | 2.50587 | 0.72212 | 12.0422 | 0.0005 | 12.254 | Site of recurrence for |
| recurrence_f | met | | | | | | | anaysis Distant met |
| Site_of_{—} | Local | 1 | 3.02239 | 0.82093 | 13.5548 | 0.0002 | 20.540 | Site of recurrence for |
| recurrence_f | | | | | | | | anaysis Local |

TABLE 27
Multivariate analysis of the clinicopathological features influencing
the outcomes in upfront surgery group for overall survival (N = 57).
Analysis of Maximum Likelihood Estimates

| | | | Parameter | Standard | Chi- | | Hazard | |
|---|---|---|---|---|---|---|---|---|
| Parameter | | DF | Estimate | Error | Square | Pr > ChiSq | Ratio | Label |
| _5M1_{——}H_score | | 1 | −0.00116 | 0.01118 | 0.0107 | 0.9176 | 0.999 | 45M1 - |
| | | | | | | | | H-score |
| CLH2_{——}H_score | | 1 | 0.00177 | 0.01131 | 0.0245 | 0.8757 | 1.002 | CLH2 - |
| | | | | | | | | H-score |
| Extracellular_{—} | Negative | 1 | −0.78471 | 0.61836 | 1.6104 | 0.2044 | 0.456 | Extracellular |
| positi | | | | | | | | positive |
| | | | | | | | | vs |
| | | | | | | | | negative |
| | | | | | | | | Negative |
| Path_diff_G1_{—} | G1-2 | 1 | −0.46908 | 0.43607 | 1.1571 | 0.2821 | 0.626 | Path |
| 2_vs_G3 | | | | | | | | diff G1- |
| | | | | | | | | 2 vs G3 |
| | | | | | | | | G1-2 |
| ADJACENT_{—} | NO | 1 | −0.32532 | 0.45251 | 0.5168 | 0.4722 | 0.722 | ADJACENT |
| ORGAN_INVAS | | | | | | | | ORGAN |
| | | | | | | | | INVASION |
| | | | | | | | | NO |
| LYMPHO | NO | 1 | −0.48694 | 0.77797 | 0.3918 | 0.5314 | 0.615 | LYMPHOVASCULAR |
| VASCULAR_{—} | | | | | | | | INVASION |
| INVAS | | | | | | | | (LVI) |
| | | | | | | | | NO |
| PERI_NEURAL_{—} | NO | 1 | −1.54236 | 0.73369 | 4.4192 | 0.0355 | 0.214 | PERI |
| INVASION | | | | | | | | NEURAL |
| | | | | | | | | INVASION |
| | | | | | | | | NO |
| Margins | Negative | 1 | −1.71666 | 0.90294 | 3.6145 | 0.0573 | 0.180 | Margins |
| | | | | | | | | Negative |
| R0_vs_R1_2 | R0 | 1 | 1.57819 | 0.94651 | 2.7801 | 0.0954 | 4.846 | R0 vs |
| | | | | | | | | R1-2 R0 |
| T_stage | T1 | 1 | −2.37275 | 1.43675 | 2.7274 | 0.0986 | 0.093 | T-stage |
| | | | | | | | | T1 |
| T_stage | T2 | 1 | 0.67797 | 0.60809 | 1.2430 | 0.2649 | 1.970 | T-stage |
| | | | | | | | | T2 |
| N0_vs_N1_N1 | N0 | 1 | −1.18377 | 0.72115 | 2.6946 | 0.1007 | 0.306 | N0 vs |
| | | | | | | | | N1-N1 |
| | | | | | | | | N0 |
| Premalignant_{—} | None | 1 | −1.17086 | 0.53021 | 4.8766 | 0.0272 | 0.310 | Premalignant |
| yes_or_{—} | | | | | | | | yes or |
| | | | | | | | | no None |
| Site_of_{—} | Distant | 1 | 1.68093 | 0.68463 | 6.0282 | 0.0141 | 5.371 | Site of |
| recurrence_f | met | | | | | | | recurrence |
| | | | | | | | | for |
| | | | | | | | | anaysis |
| | | | | | | | | Distant |
| | | | | | | | | met |
| Site_of_{—} | Local | 1 | 2.08405 | 0.79937 | 6.7971 | 0.0091 | 8.037 | Site of |
| recurrence_f | | | | | | | | recurrence |
| | | | | | | | | for |
| | | | | | | | | anaysis |
| | | | | | | | | Local |

TABLE 28
Site of metastasis and corresponding biopsy sites tissue.

| Distant metastases (N) | Biopsy site provided for MUC5AC testing |
|---|---|
| Liver (3) | Liver |
| Liver (1) | Pancreas |
| Lung (1) | Lung |
| Liver, peritoneum, and ovary (1) | Ovary |
| Peritoneum and small bowel (2) | Peritoneal lesion −1 |
| | Small bowel −1 |
| Peritoneum (1) | Peritoneal lesion |
| Lung and adrenal gland with PDA invading | Bladder |
| the bladder (1) | |

TABLE 29
Comparing MUC5AC distribution in primary and metastatic tissues

| | 45M1 | CLH2 | EC-45M1 |
|---|---|---|---|
| | Metastatic sites (N = 9) vs. resected primary sites (N = 100) (p-value) | | |
| H-score | 199 vs. 149 | 196 vs. 145 | 199 vs. 132 (0.02) |
| | (0.05) | (0.04) | |
| % distribution | 68 vs. 50 (0.03) | 67 vs. 50 (0.03) | NA |
| Positive | 100 vs. 96* | 100 vs. 96* | 100 vs. 72 (0.06) |
| percentage | | | |

1. A method of treating a pancreatic lesion in a subject, the method comprising:
a) collecting a tissue sample from the subject,
b) detecting Mucin-5AC (MUC5AC) levels in the tissue sample,
c) classifying the subject as having a benign pancreatic lesion, a pre-cancerous pancreatic lesion, or a malignant pancreatic lesion, wherein:
i.the benign pancreatic lesion will not express MUC5AC;
ii.the pre-cancerous pancreatic lesion comprises increased expression of the immature MUC5AC, the mature MUC5AC, or a combination thereof, relative to a healthy control or the benign pancreatic lesion; and
iii.the malignant pancreatic lesion comprises increased expression of the immature MUC5AC, mature MUC5AC, or a combination thereof, relative to the benign pancreatic lesion or the pre-cancerous pancreatic lesion, and
d) treating the subject classified as having the benign pancreatic lesion by monitoring said subject for progression or regression of the pancreatic lesion,
e) treating the subject classified as having the pre-cancerous pancreatic lesion by monitoring said subject for progression or regression of the lesion, or surgical resection of the pancreatic lesion, and
f) treating the subject classified as having the malignant pancreatic lesion with at least one anti-cancer therapy comprising a chemotherapeutic agent, an imaging modality, or surgical resection.

2. The method of 1, wherein the benign pancreatic lesion comprises a low-risk for pancreatic ductal adenocarcinoma (PDA).

3. The method of claim 1, wherein the pre-cancerous pancreatic lesion comprises a moderate-risk for PDA.

4. The method of claim 1, wherein the malignant pancreatic lesion comprises a high-risk for PDA.

5. The method of claim 1, wherein the malignant pancreatic lesion comprises a PDA.

6. The method of claim 1, wherein MUC5AC levels are scored from 0 to 300.

7. The method of claim 1, wherein the benign pancreatic lesion comprises a MUC5AC score of 50 or less.

8. The method of claim 1, wherein the pre-cancerous pancreatic lesion comprises a MUC5AC score of 100 or less.

9. The method of claim 1, wherein the malignant pancreatic lesion comprises a MUC5AC score greater than 100.

10. The method of claim 1, wherein the healthy control comprises a MUC5AC score less than 10.

11. The method of claim 1, wherein the sample comprises a blood sample, a serum sample, or a plasma sample.

12. The method of claim 1, wherein MUC5AC is detected by an antibody, or a fragment thereof, an enzyme linked immunosorbent assay (ELISA), a western blot assay, mass spectrometry, RNA sequencing, real-time polymerase chain reaction (RT-PCR), or a derivative thereof.

13. The method of claim 12, wherein the antibody, or fragment thereof, comprises a monoclonal antibody selected from CLH2, 45M1, 2-11M1, Nd2, or a fragment thereof.

14. The method of claim 1, further comprising detecting carbohydrate antigen 19-9 (CA19-9), a methylation biomarker, a blood biomarker, or a combination thereof.

15. The method of claim 14, wherein the methylation marker comprises ADAMTS1, ADAMTS22, ALX4, APC, BMP3, BNC1, BRCA1, CCND2, CDKN1C, CUX2, DAPKI, DCC, EPB41L3, ESR1, FAM150A, FSD1, GPC3, HIC1, HIST1H4E, HOXA1, MAPT, LOC100128977, LOC100130148, MESTv2, MIR663, MUC2, MYF3, MYOD1, NPTX2, p14, p16, PCDH10, PENK, PGKI, PGR-dist, PGR-prox, PLAU, ppENK, RARB, RASSF1A, REGlA, RUNX3, SARP2, SEMA5A, SEPT9v2, SFRP1, SFRP2, SIX3, SOCS1, SPARC, SPSB4, SRBC, SST, SYK, TAC1, TBX3, TFPI2, THBS1, TMS, TRIM73, TSPAN2, UCLH1, VHL, WNT5A, ZNF154, ZNF695, hMLH1, CDKN2B, RB1, or a combination thereof.

16. The method of claim 14, wherein the blood biomarker comprises a mutation to one or more genes selected from KRAS, p53, CDK2NA, ATM, and PIK3CA.

17. The method of claim 1, wherein the chemotherapeutic agent comprises fluorouracil (5FU), cisplatin, irinotecan, oxaliplatin, gemcitabine (Gem), nab-paclitaxel, leucovorin, or a combination thereof.

18. The method of claim 1, wherein the imaging modality comprises magnetic resonance imaging (MRI), computed tomography (CT), positron emission tomography (PET), endoscopic ultrasound (EUS), abdominal ultrasound, or a combination thereof.

19. A method of decreasing expression of Mucin-5AC (MUC5AC) in a malignant pancreatic cell in a subject diagnosed with pancreatic ductal adenocarcinoma (PDA), the method comprising:
a) collecting a tissue sample from the subject,
b) detecting an expression level of MUC5AC in the sample relative to a control sample, and
c) treating the subject with at least one anti-cancer therapy, wherein the anti-cancer therapy reduces the PDA and the expression level of MUC5AC in the subject relative to the control.

20. The method of claim 19, wherein the expression level of MUC5AC is scored from 0 to 300.

21-28. (canceled)

29. A method of preventing maturation of Mucin-5AC (MUC5AC) in a pancreatic cell in a subject diagnosed with a pancreatic lesion, the method comprising:
a) collecting a tissue sample from a subject,
b) detecting an immature MUC5AC, a mature MUC5AC, or a combination thereof, in the tissue sample, and
c) treating the subject with PDA with at least one anti-cancer therapy, wherein the anti-cancer therapy prevents the immature MUC5AC from converting into the mature MUC5AC or an immunogenic MUC5AC, or wherein the anti-cancer therapy prevents the mature MUC5AC from converting into the immunogenic MUC5AC.

30-65. (canceled)
